(19) United States
(12) Patent Application Publication
(10) Pub. No.: US 2026/0188478 A1
(43) Pub. Date: Jul. 2, 2026

(54) METHODS AND APPARATUS FOR RESPIRATORY THERAPY DEVICE CONNECTIVITY

(71) Applicant: ResMed Sensor Technologies Limited, Sandyford, Dublin (IE)

(72) Inventors: Chris MERJANE, Sydney (AU); Amila Jeewaka FERNANDO, Sydney (AU); Romain Geoffrey Pierre DOUMENC, Clonskeagh, Dublin (IE); Aoibhe Jacqueline TURNER-HEANEY, Clonskeagh, Dublin (IE); Shahil MAHARAJ, Clonskeagh, Dublin (IE); Alexander STEWART, Clonskeagh, Dublin (IE); Léa LOUESDON, Clonskeagh, Dublin (IE)

(73) Assignee: ResMed Sensor Technologies Limited, Sandyford, Dublin (IE)

(21) Appl. No.: 18/998,155

(22) PCT Filed: Sep. 7, 2023
(86) PCT No.: PCT/EP2023/074665

§ 371 Date: Jan. 24, 2025

Related U.S. Application Data
(60) Provisional application No. 63/405,191, filed on Sep. 9, 2022.

(51) Int. Cl. G16H 40/63; H04W 12/03; H04W 12/06; H04W 84/12

(52) U.S. Cl. CPC G16H 40/63; H04W 12/03; H04W 12/06; H04W 84/12

(57) ABSTRACT

A wireless communications enabled memory card (100) has a card interface (104) for wired communicating with a medical device. The memory card has a non-volatile memory (106) for storing therapy data provided by the medical device. The memory card may have a Wi-Fi transceiver (110) for joining a Wi-Fi network. The memory card may receive authentication information for joining the network from the medical device via the card interface or from a wireless device via a direct Wi-Fi connection. After joining the Wi-Fi network, the memory card may transmit the therapy data to a remote server (130).

### 1 CROSS REFERENCE TO RELATED APPLICATIONS

This application claims the benefit of U.S. Provisional Patent Application No. 63/405,191, filed Sep. 9, 2022, the entire content of which is incorporated herein by reference.

### BACKGROUND OF THE TECHNOLOGY

### 1.1 Field of the Technology

The present technology generally relates to transmitting use and/or medical information, such as high-resolution data, from a respiratory therapy device to a remote server. Some examples of the technology implement a wireless memory card mountable on the respiratory therapy device, and capable of joining a Wi-Fi network to transmit high resolution data captured by the respiratory therapy device to the remote server. Some examples of the technology enable the respiratory therapy device to export therapy data via one or more barcodes which are scannable by a wireless device for transmission to the remote server.

### 1.2 Description of Related Art

Home-based respiratory therapy devices allow patients to receive respiratory treatments at the comfort of the patients' home. To check for compliance or to monitor conditions of the patients, clinicians need to regularly review therapy data collected by the respiratory therapy devices.

Some existing respiratory therapy devices implement a cellular modem to transfer therapy data to a remote server that is accessible by the clinician. However, communication limitations such as due to quality or availability of such networks, the cellular modem is not always a reliable device for transferring data such as high resolution data. For example, a respiratory therapy device may have collected an extensive quantity of pressure and flow data samples from one or more sensors over an eight-hour treatment session, which may be collected for weeks and months. If pressure sensors sample pressure values in a range of 100 to 250 Hertz (Hz) (which may be lower or higher), over the course of just one night of sleep (e.g., 8 hours), such a session could accumulate as much as 7,200,000 pressure samples. If additional sensors sample at similar rates, that number would multiply, such as double (e.g., a flow sensor), triple (e.g., a humidity sensor), quadruple, e.g., a temperature sensor), etc. With a low quality cellular bandwidth limitation, the respiratory therapy device may be limited to transfer low resolution data, such as a summary or subset of the samples, as opposed to sending all or a substantial portion of the samples collected over the eight-hour treatment session.

Other existing respiratory therapy devices are not equipped with a cellular modem. They rely on a standard secure digital (SD) memory card to record therapy data. To export therapy data, a user or patient needs to manually pull the SD card out of the respiratory therapy device, physically carry the SD card to the clinician's office and insert the card into a computer or terminal at a physician or clinician's office.

In view of the foregoing, there is a need for a cost effective, time-saving communications approach to data transfer from a home therapy device such as to support high resolution data transmission to a remote server, and simplify the process for exporting data to the remote server. The technology disclosed herein aims at providing solutions that may transmit high resolution data to the remote server with minimal user intervention.

### 2 BRIEF SUMMARY OF THE TECHNOLOGY

The present technology is directed towards transmitting high resolution data from a medical device to a remote server.

Some implementations of the present technology may include a memory card. The memory card may include a card interface configured to communicate with a medical device. The memory card may include a non-volatile memory configured to store authentication information to join a wireless network. The non-volatile memory may be configured to store therapy data provided by the medical device. The memory card may include a network interface including at least one wireless communication transceiver. The memory card may include one or more processors. The one or more processors may be configured to instruct the at least one wireless communication transceiver to join the wireless network using the authentication information. The one or more processors may be configured to instruct the at least one wireless communication transceiver to access and wirelessly transmit the therapy data stored in the memory to a remote server via the wireless network.

In some implementations, the authentication information may be received from the medical device via the card interface, and stored in the non-volatile memory after being received from the medical device. The at least one wireless communication transceiver may be configured to wirelessly receive the authentication information from a wireless device. The at least one wireless communication transceiver may include a wireless fidelity (Wi-Fi) communication transceiver. The one or more processors may be configured to encrypt the data provided by the medical device, and/or instruct the at least one wireless communication transceiver to wirelessly transmit the encrypted data to the remote server via the wireless network. The card interface may be configured to be physically and operably engaged with the medical device. The authentication information to join the wireless network includes a wireless network name and a password to access the wireless network. The one or more processors may be configured to instruct the at least one wireless communication transceiver to access the remote server using credential information. The credential information includes a device identifier of the medical device. The credential information may be obtained by receiving the credential information from the medical device via the card interface. The credential information may be obtained by wirelessly receiving the credential information from a wireless device. The credential information may be obtained by retrieving the credential information from a firmware of the memory card. The one or more processors may be configured to detect new data being written into the non-volatile memory. The one or more processors may be configured to instruct the at least one wireless communication transceiver to wirelessly transmit the new data to the remote server via the wireless network.

Some implementations of the present technology may include a method. The method may include receiving, via a card interface of a memory card, therapy data provided by a medical device. The method may include storing, in a non-volatile memory of the memory card, the received therapy data. The method may include instructing, by one or more processors of the memory card, at least one wireless communication transceiver to join a wireless network using authentication information. The method may include instructing, by the one or more processors of the memory card, the at least one wireless communication transceiver to wirelessly transmit the therapy data stored in the non-volatile memory to a remote server via the wireless network.

In some implementations, the method may include receiving the authentication information from the medical device via the card interface. The method may include wirelessly receiving, by the at least one wireless communication transceiver, the authentication information from a wireless device. The at least one wireless communication transceiver may include a wireless fidelity (Wi-Fi) communication transceiver. The method may include instructing, by the one or more processors of the memory card, the at least one wireless communication transceiver to access the remote server using credential information. The method may further include receiving the credential information from the medical device via the card interface. The method may further include wirelessly receiving the credential information from a wireless device. The method may further include retrieving the credential information from a firmware of the memory card.

Some implementations of the present technology may include a method of data reading. The method may include reading a first barcode output by a medical device, the first barcode encoding information of the medical device. The method may include registering, by one or more processors, the medical device with a remote server based on the first barcode. The method may include reading a second barcode output by the medical device, the second barcode encoding therapy data recorded by the medical device. The method may include wirelessly transmitting the second barcode or the therapy data to the remote server.

In some implementations, the method may further include decoding the first barcode to obtain the information of the medical device. The method may further include transmitting, by a wireless transceiver, the decoded information of the medical device to the remote server. The first barcode may be further configured to encode an encryption key. The information of the medical device may include at least one of the following: a device identifier and/or one or more device settings. The device settings may include one or more of the following: therapy mode, maximum pressure, minimum pressure, and expiratory pressure relief (EPR) pressure. The method may further include decoding the first barcode to obtain the information of the medical device and the encryption key. The method may further include encrypting the information of the medical device by using the encryption key. The method may further include transmitting, by a wireless transceiver, the encrypted information of the medical device to the remote server. Each of the first barcode and the second barcode may be a two-dimensional code.

In some implementations, the method may further include decoding the second barcode to obtain the therapy data recorded by the medical device. The method may further include wirelessly transmitting the therapy data to the remote server after decoding the second barcode. The method may further include displaying a prompt to a user to scan the second barcode according to a predetermined schedule. The method may further include determining, by the one or more processors, whether the second barcode has been scanned according to a predetermined schedule. The method may further include displaying a prompt to a user to scan the second barcode when the second barcode has not been scanned according to the predetermined schedule. The medical device may be a respiratory pressure medical device. The therapy data may include one or more of the following: one or more respiratory parameters of a user as collected by the medical device, usage data of the medical device, and one or more device settings of the medical device. The method may further include decoding the second barcode to obtain the therapy data recorded by the medical device. The method may further include encrypting the therapy data by using the encryption key obtained from the first barcode. The method may further include wirelessly transmitting the encrypted therapy data to the remote server.

Some implementations of the present technology may include a method for reporting therapy data of a medical device to a remote server. The method may include transforming information of the medical device to a first barcode. The method may include outputting, for display, the first barcode for registering the medical device with a remote server. The method may include transforming therapy data recorded by the medical device to a second barcode. The method may include outputting, for display, the second barcode for transmitting the therapy data to the remote server.

In some implementations, the information of the medical device transformed to the first barcode may include one or more of the following: a serial number, one or more device settings, and an encryption key of the medical device. The device settings may include one or more of the following: therapy mode, maximum pressure, minimum pressure, and expiratory pressure relief (EPR) pressure. Each of the first barcode and the second barcode may be a two-dimensional code. Of course, portions of the aspects may form sub-aspects of the present technology. Also, various ones of the sub-aspects and/or aspects may be combined in various manners and also constitute additional aspects or sub-aspects of the present technology.

Other features of the technology will be apparent from consideration of the information contained in the following detailed description, abstract, drawings and claims.

### 2.1 BRIEF DESCRIPTION OF DRAWINGS

The present technology is illustrated by way of example, and not by way of limitation, in the figures of the accompanying drawings, in which like reference numerals refer to similar elements including:

FIG. 1A shows an example environment in which a wireless memory card receives Wi-Fi authentication from a wireless device through a medical device;

FIG. 1B shows an example environment in which the wireless memory card receives Wi-Fi authentication from the wireless device via a direct Wi-Fi connection;

FIG. 2A shows an example environment in which the memory card receives server credential and Wi-Fi authentication from the medical device;

FIG. 2B shows an example environment in which the memory card receives server credential from a medical device and receives Wi-Fi authentication from a wireless device;

FIG. 2C shows an example environment in which the memory card receives Wi-Fi authentication and server credential from the wireless device via a medical device;

FIG. 2D shows an example environment in which the memory card receives Wi-Fi authentication and server credential from the wireless device via a direct Wi-Fi connection;

FIG. 3 shows a flow diagram of transmitting data to the remote server;

FIG. 4 shows block diagrams of the medical device and the wireless device;

FIGS. 5A-5L are screenshots of a graphic user interface of the wireless device showing processes to obtain data from the medical device through barcode scanning;

FIG. 6 illustrates a flow diagram of an example data reading process performed by the wireless device 140;

FIG. 7 illustrates a flow diagram of a process for reporting therapy data from the medical device to the remote server.

FIG. 8A shows an example system in accordance with the present technology. A patient 1000 wearing a patient interface 3000 receives a supply of pressurised air from an RPT device 4000. Air from the RPT device 4000 is humidified in a humidifier 5000, and passes along an air circuit 4170 to the patient 1000. A bed partner 1100 is also shown.

FIG. 8B shows an RPT device 4000 in use on a patient 1000 with a nasal mask 3000.

FIG. 8C shows an RPT device 4000 in use on a patient 1000 with a full-face mask 3000.

FIG. 9 shows an example non-invasive patient interface 3000 in the form of a nasal mask.

FIG. 10A shows an RPT device 4000 in accordance with one form of the present technology.

FIG. 10B shows a schematic diagram of the pneumatic circuit of an RPT device 4000 in accordance with one form of the present technology. The directions of upstream and downstream are indicated.

FIG. 10C shows a schematic diagram of the electrical components of an RPT device 4000 in accordance with one aspect of the present technology.

FIG. 10D shows a schematic diagram of the algorithms 4300 implemented in an RPT device 4000 in accordance with an aspect of the present technology. In FIG. 10D, arrows with solid lines indicate an actual flow of information, for example via an electronic signal.

FIG. 10E is a flow chart illustrating a method 4500 carried out by the therapy engine module 4320 of FIG. 10D in accordance with one aspect of the present technology.

FIG. 11 shows a humidifier 5000.

### 3 DETAILED DESCRIPTION OF EXAMPLES OF THE TECHNOLOGY

Before the present technology is described in further detail, it is to be understood that the technology is not limited to the particular examples described herein, which may vary. It is also to be understood that the terminology used in this disclosure is for the purpose of describing only the particular examples discussed herein, and is not intended to be limiting.

The following description is provided in relation to various examples which may share one or more common characteristics and/or features. It is to be understood that one or more features of any one example may be combinable with one or more features of another example or other examples. In addition, any single feature or combination of features in any of the examples may constitute a further example.

### 3.1 Wireless Memory Card

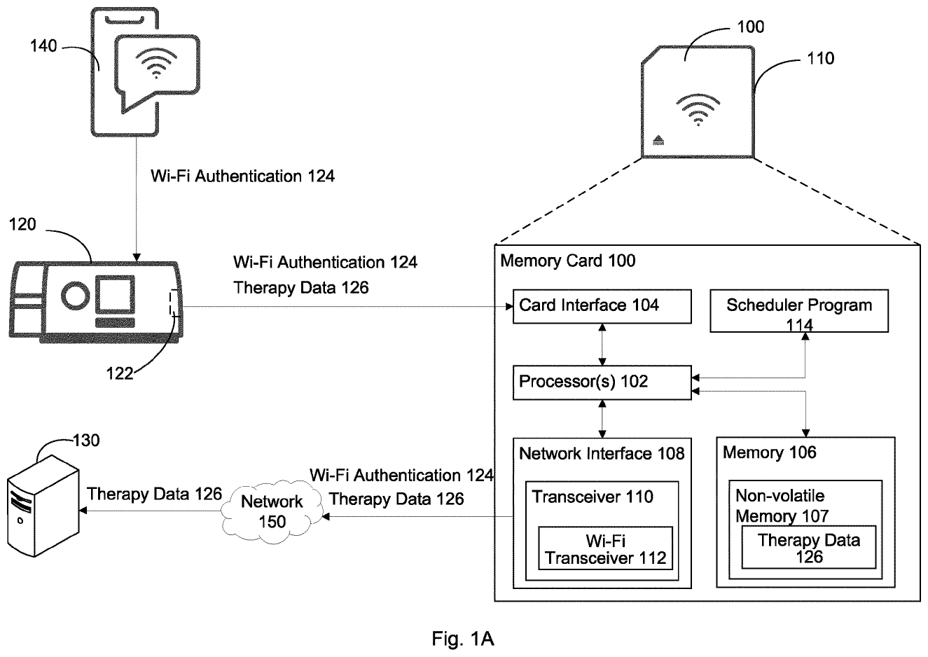

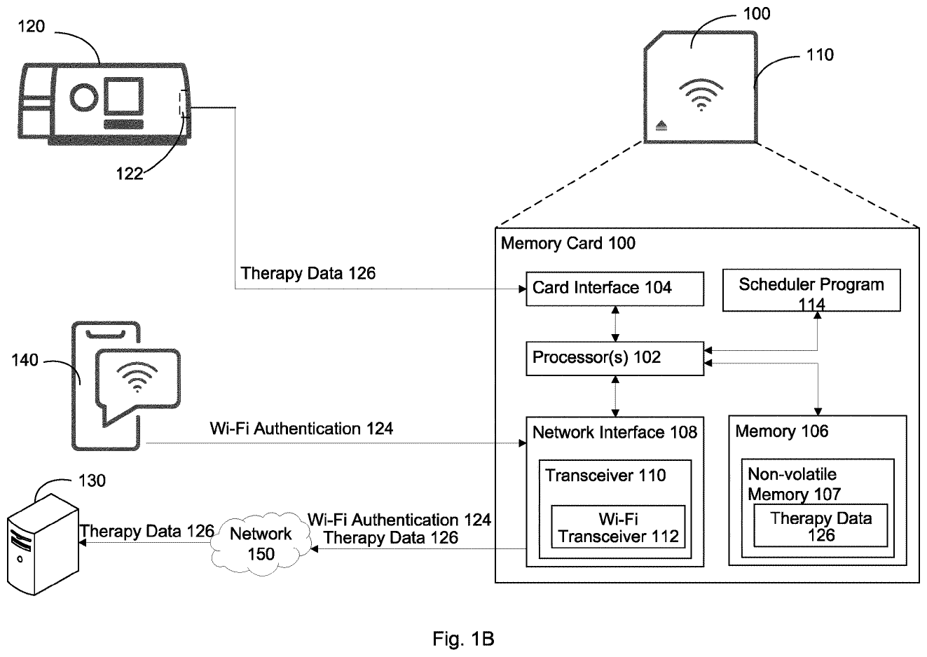

One aspect of the present technology relates to a wireless memory card mountable on a medical device, and capable of joining a communications network such as a wireless fidelity (Wi-Fi) network to transmit high resolution data captured by the medical device to a remote server. Implementation of such a memory card may be considered in relation to FIGS. 1A through 3.

FIG. 1A illustrates an environment in which a wireless memory card 100 may interact with a medical device 120, a remote server 130, and a wireless device 140.

The medical device 120 may be a respiratory therapy device that provides respiratory treatment to a user, such as an RT or RPT described herein. The medical device 120 may have a memory card slot 122 for removably accepting the memory card 100. In one embodiment, the medical device 120 may be a respiratory pressure therapy (RPT) device and/or a high flow therapy device (HFT). The medical device 120 may provide a flow of breathable gas to the user. An interface, such as a mask, may be used to interface the medical device 120 to the user. Depending upon the therapy to be applied, the interface may form a seal, e.g., with a face region of the user, to facilitate the delivery of gas at a pressure at sufficient variance with ambient pressure to effect therapy.

The remote server 130 may be a remotely located computing system that collects therapy data, such as use data, measured/determined health parameters or events, of the medical device 120 such as a plurality of such devices. The remote server 130 may be implemented to monitor conditions or treatment progress of the user based on data of the medical device 120. The remote server 130 may be a cloud-based server system, and may be implemented as one or more servers such as to divide the functionality amongst such devices. The remote server 130 may be accessible to a clinician(s). In some implementations, the remote server may merely receive and process data, and another system may receive or access such processed data and generate or provide insights such as by providing clinician access to such processed data and insights.

The wireless device 140 may be a computing system accessible by a user. Examples of the wireless device 140 may include mobile phone, tablet, netbook, desktop computer, laptop computer, and wearable computing device such as a smartwatch, among other possibilities.

The memory card 100 may have a housing 110 that is insertable into the medical device 120. The housing 110 may be configured to be removably accepted by the memory card slot 122 of the medical device 120. The memory card 100 may slide in and out of the memory card slot 122 to engage or disengage respectively with a data communications interface of the medical device 120. The memory card 100 may be dimensioned and configured as any of a removable flash memory card, among other possibilities.

The memory card 100 may include one or more built-in processors 102, a card interface 104 for communicating with the medical device 120 (i.e., via the data communications interface of the medical device 120), memory 106, and a network interface 108. The memory 106 may include a non-volatile memory 107 for storing data received from the medical device 120.

The card interface 104 may be configured to be physically and operably engaged with the data communications interface of the medical device 120. The card interface 104 may be a communication and power-supply interface. The processor(s) 102, the memory 106 and the network interface 108 may receive power from the medical device 120 via the card interface 104 of the card and data communications interface of the medical device 120. The card interface 104 may include, for example, any of the following formats: secure digital (SD), compact flash (CF), multimedia card (MMC), memory stick (MS) and universal serial bus (USB). In one example, the memory card 100 may be an SD card, and the memory card slot 122 may include an SD memory card slot.

The medical device 120 may routinely write data onto the memory 106, such as the non-volatile memory 107, of the memory card 100 via the card interface 104. Such data may include therapy data 126 related to one or more treatment sessions of the user. Therapy data 126 may include, but not limited to, one or more sensor measurements or determined information or parameters, such as any from the pre-processing module 4310 and/or the therapy engine module 4320, of the user as collected by the medical device 120, usage data of the medical device 120, and one or more device settings of the medical device 120 used in the user's treatment session(s). The therapy data may include respiratory measurements such as how many respiratory events a user has experienced, such as number of respiratory events per hour. Therapy data 126 may include usage data such as how much time the medical device has been used such as usage hours, and how much time a mask has been worn by the user during one or more treatment sessions, number of times that the mask is on and off the user, and efficiency of mask seal, among others. The therapy data may include device settings such as one or more of the following parameters of the medical device: mode, maximum pressure, minimum pressure, and expiratory pressure relief (EPR) pressure, among other possibilities. With such data in the memory card 100, a clinician may optionally access the memory card, such as by removal of it from the medical device 120 and insertion into a computer system, such as a laptop or other reader enabled computer, to review the data. Optionally with such a reader enabled computer, the physician may input new device settings into the memory card, which in turn can be accessed/read by, and applied for controlling operations of, the medical device 120 when the memory card 100 is inserted therein. Optionally, such access to data and/or input of device settings may be performed from a remote clinician computer, such as by transferring such data through a communications interface of the memory card.

For example, the network interface 108 may include at least one wireless communication transceiver 110, which may include a Wi-Fi communication transceiver 112. The transceiver 110 may include an antenna adapted to wirelessly transmit and receive data packets. The network interface 108 may be configured to detect potential wireless networks to which the memory card may be connected.

### 3.1.1 Network Authentication

The network interface 108 may enable the memory card 100 to connect to a network 150, such as the user's home Wi-Fi network. The memory card 100 may be configured with authentication information to log into the network 150 through a one-off upfront setup process. The authentication information may include, for example, a wireless network name or service set identifier (SSID), and may include a password for the network 150.

There are several ways to set up the memory card 100 so as to provide it with the authentication information for the wireless network. In some implementations, as illustrated in FIG. 1A, authentication information may be provided to the memory card 100 by the medical device 120 via the card interface 104. In such as case, the medical device 120 may receive the authentication information from the wireless device 140 and/or a user entering it into a user interface such as of the medical device 120 or wireless device 140. In some implementations, as illustrated in FIG. 1B, authentication information may be provided to the memory card 100 more directly from the wireless device 140 via a direct Wi-Fi connection between the memory card 100 and wireless device 140. Details of such processes are provided below.

For example, referring to FIG. 1A, in the first example, a Bluetooth connection may be set up between the medical device 120 and the wireless device 140. The wireless device 140 may prompt a user to enter the authentication information of the network 150, shown as Wi-Fi authentication 124 in FIG. 1A. The wireless device 140 may include an application that prompts the user to enter the authentication information. Alternatively, a browser on the wireless device 140 may display a web page that prompts the user to enter the authentication information. Once the user enters the authentication information into the wireless device 140, the wireless device 140 may transmit the authentication information to the medical device 120 via the Bluetooth connection. The medical device 120 may then write the authentication information onto the memory card 100 via the card interface 104. The authentication information may be stored in the memory card 100, such as in the non-volatile memory 107, and be used by the memory card 100 for connecting to the network 150.

In another example, as illustrated in FIG. 1B, the network interface 108 of the memory card 100 may be configured with a server application to provide a Wi-Fi direct capability, allowing a direct peer-to-peer Wi-Fi connection with the wireless device 140. For example, after the memory card 100 is inserted into the medical device 120, the memory card 100 may get powered by the medical device 120 and start broadcasting a Wi-Fi network of the memory card 100. The wireless device 140 may than connect to the Wi-Fi network broadcast by the memory card 100, and form a direct Wi-Fi connection with the memory card 100. Thereafter, the wireless device 140 may prompt the user to enter the authentication information of the network 150, either through an application or a web page displayed in a browser such as using the server application of the memory card 100. Once the user enters the authentication information into the wireless device 140, the wireless device 140 may transmit the authentication information to the memory card 100 via the direct Wi-Fi connection. The Wi-Fi transceiver 112 may then receive the authentication information transmitted by the wireless device 140. The authentication information may then be stored in the memory card 100, and used by the memory card thereafter for establishing another Wi-fi connection with access to the network 150.

Thus, once the memory card 100 has the authentication information, the Wi-Fi transceiver 112 may use the authentication information to establish a connection(s) with the network 150 using a Wi-fi link to the network 150.

### 3.1.2 Data Transmission to Remote Server

As discussed in more detail herein, periodically, the medical device 120 will store or write therapy data 126 to the memory card 100. After the medical device 120 writes onto the memory card 100 data associated with the user's treatment session(s), the memory card 100, according to its programming instructions and processor(s) 102, may then transmit such data to the remote server 130 via the network 150. In this regard, the memory 106 may store an address of the remote server 130. The address of the remote server 130 may be, for example, a domain name, a global Internet protocol (IP) and/or a media access control (MAC) address for such access. The transceiver 110, such as the Wi-Fi Transceiver 112, may wirelessly transmit data stored in the memory to the address of the remote server 130. Optionally, in some versions, the memory card 100 may be pre-configured with a black listing protocol that limits the card's communications to only a desired server(s) (i.e., remote server 130) so as to preclude it from connecting with or communicating with other servers (e.g., non-authentic servers) that are not part of the system.

### 3.1.2.1 Secured Access

The remote server 130 may be a secure server having restricted access, requiring credential information to access the remote server 130. Credential information may, for example, include a device identifier of the medical device 120 or a device identifier of a card, such as a memory card or wireless memory card, or a combination of both. The device identifier(s) may be unique, such as a non-sequential unique number, enabling the remote server 130 to determine the medical device 120 and/or memory card 100 that generates the data when the remote server 130 receives the data from the memory card 100. The device identifier(s) may include one or more of the following: a serial or other unique number of the medical device 120, a type or device model of the medical device 120, a serial number or other unique number of the memory card 100, and a type of device model of the memory card 100.

In some implementations, the medical device 120 may have the credential information for accessing the remote server 130. The medical device 120 may transfer the credential information to the memory card 100. The processor(s) 102 of the memory card may receive the credential information from the medical device 120 via the card interface 104, and store the credential information in the memory.

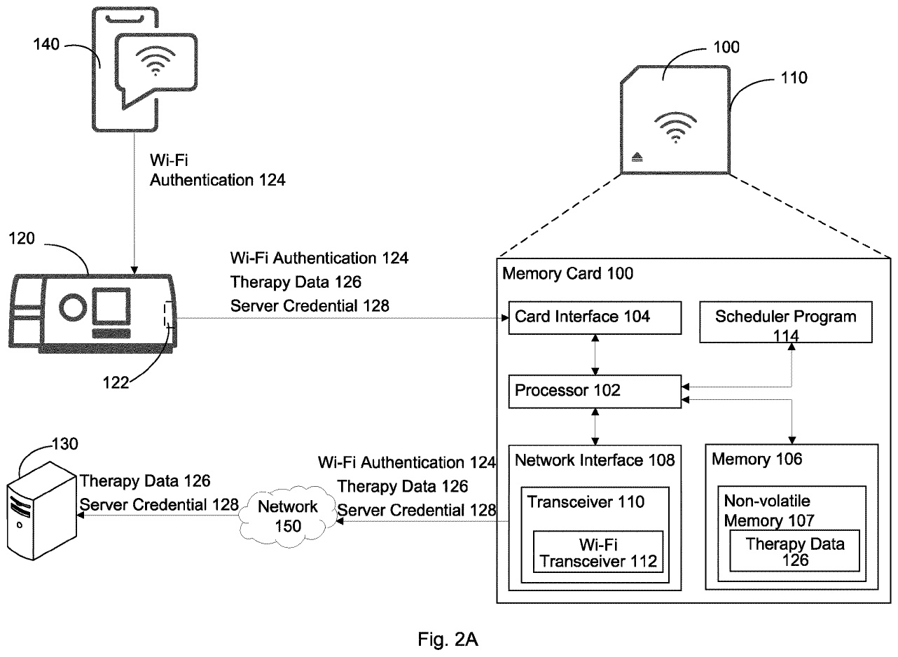

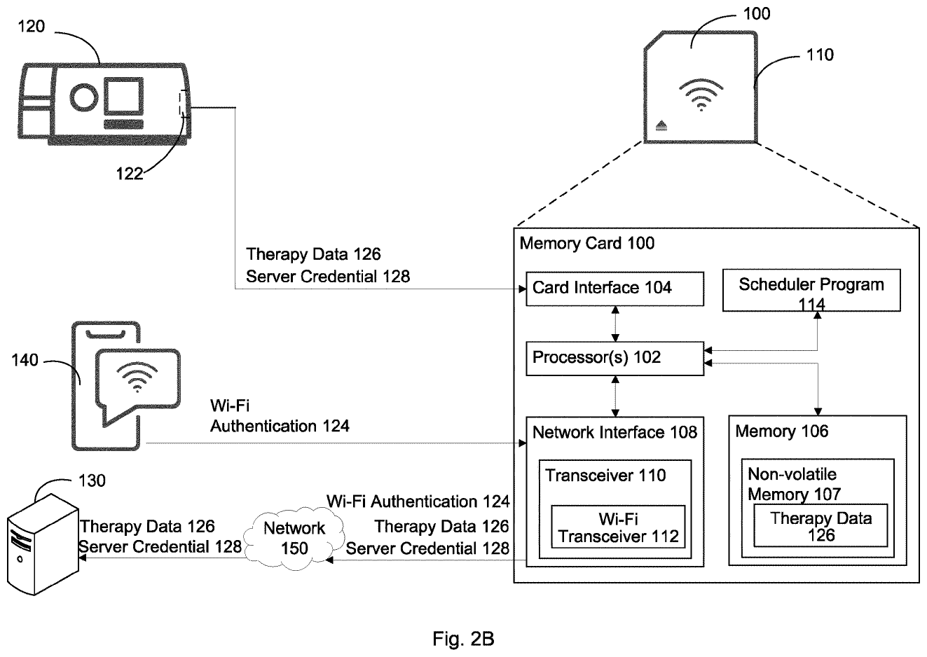

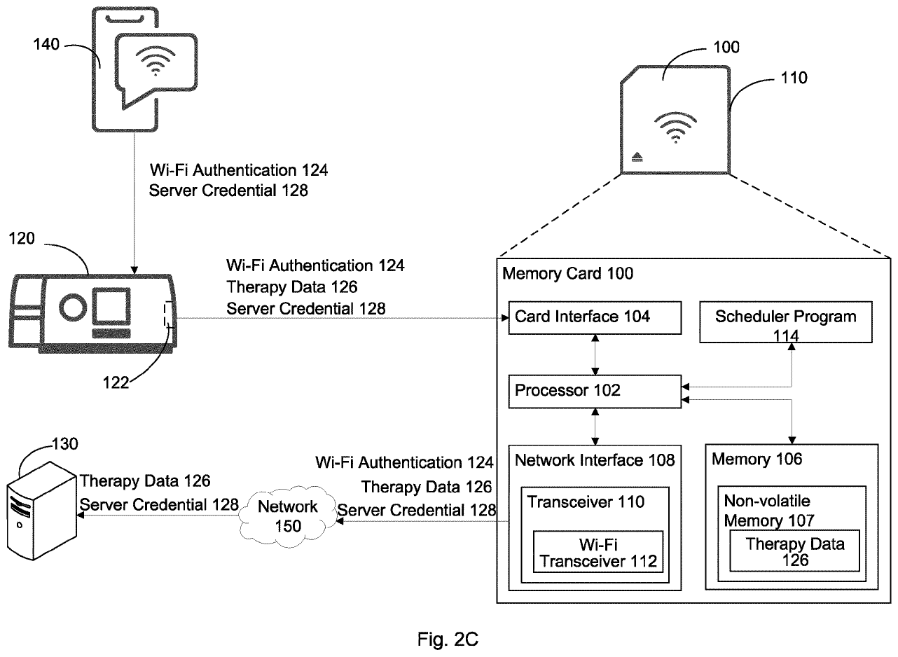

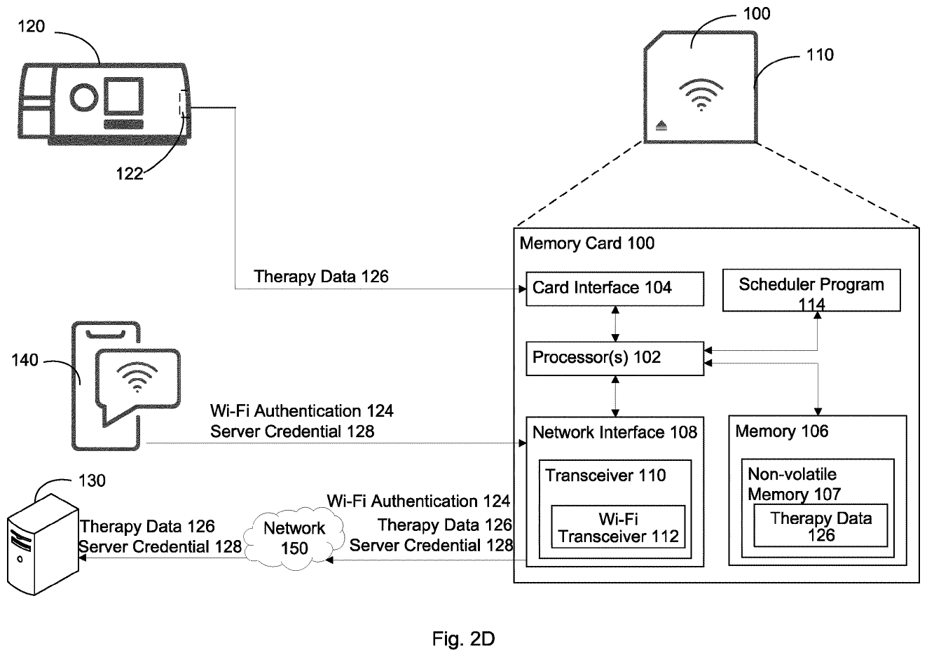

In some implementations, such as the example as shown in FIG. 2A, the medical device 120 may send credential information (shown as server credential 128), authentication information (shown as Wi-Fi authentication 124) and therapy data 126 to the memory card 100 via the card interface 104. The memory card 100 may use the authentication information to log onto or access the network 150, and log into or access the remote server 130 using the credential information. After accessing the remote server 130, the memory card 100 may upload the therapy data 126 to the remote server 130. The credential information may then serve to identify source of the therapy data 126. Optionally, the remote server 103 may send one or more device settings (e.g., therapy setting or parameter such as a treatment pressure etc. or other described herein) to the memory card, which in turn may be accessed/read by, and applied for controlling operations of, the medical device 120.

In another example as shown in FIG. 2B, the medical device 120 may send credential information (e.g., server credential 128), and therapy data 126 to the memory card 100 via the card interface 104. The wireless device 140 may send the authentication information (e.g., Wi-Fi authentication 124) to the memory card 100 via the direct Wi-Fi connection. The memory card 100 may use the authentication information to log onto or access the network 150, and access the remote server 130 using the credential information, and upload the therapy data 126 to the remote server 130.

In some implementations, the wireless device 140 may have the credential information for accessing the remote server 130. In one example as shown in FIG. 2C, when the wireless device 140 sends the authentication information (e.g., Wi-Fi authentication 124) to the medical device 120, it may also send the credential information (e.g., server credential 128). The medical device 120 may write the Wi-Fi authentication information, the credential information and the therapy data 126 to the memory 106, such as together when the medical device 120 periodically sends therapy data 126 to the memory 106.

In another example as shown in FIG. 2D, the wireless device 140 may wirelessly transmit the credential information (e.g., server credential 128) and the authentication information (e.g., Wi-Fi authentication 124) to the memory card 100, so that the memory card 100 may wirelessly receive the credential information directly from the wireless device 140.

In yet another embodiment, the credential information may be hardcoded or otherwise be provided in the memory card 100. For example, the firmware in the memory card 100 may have the credential information for accessing the remote server 130. As such, neither the medical device 120 nor the wireless device 140 needs to provide or transmit the credential information to the memory card 100.

### 3.1.2.2 Non-Secured Access

In some implementations, the remote server 130 may be a non-secure server such that the server does not require server credentials for transferring data to the server. In such an implementation the server may only require credentials for accessing the data once on the server. In some such cases, the memory card 100 may perform bulk data transfer to the remote server 130, without providing any credential information to the remote server 130. Thus, the memory card 100 may freely wirelessly transmit therapy data in bulk transfers to the server.

### 3.1.3 Encryption

In some implementations, the memory card 100 may send encrypted data to the remote server 130, so as to have secure communications with the remote server 130.

In one example, the memory card 100 may have stored an encryption key to encrypt any data for sending to the remote server 130. The processor(s) 102 may encrypt data provided by the medical device 120 using the encryption key, and instruct the transceiver 110, such as the Wi-Fi transceiver 112, to wirelessly transmit the encrypted data to the remote server 130.

In another example, the memory card 100 may include an encrypting circuit that encrypts data stored in the memory 106. The encrypting circuit may be provided in an integrated circuit chip.

In yet another example, the medical device 120 may perform encryption, and send encrypted data to the memory card 100 for storage. The memory card 100 may then transmit the encrypted data to the remote serve 130, without performing additional encryption or decryption.

### 3.1.4 Scheduler Program

The memory card 100 may a scheduler program 114, executed by the processor(s) 102. The scheduler program may be software or firmware that includes the rules of the processor(s) 102 for controlling or instructing what data to transmit, when and/or where to transmit the data of the memory 106.

In one example, the scheduler program 114 may periodically arrange transferring data written in the non-volatile memory 107 to the remote server 130.

In another example, the scheduler program 114 may detect new data written into the non-volatile memory 107. For example, when a new file appears in the non-volatile memory 107, the scheduler program 114 may automatically instruct the transceiver 110, such as the Wi-Fi transceiver 112, to transfer the new file to the remote server 130 via the network 150.

In yet another example, the scheduler program 114 may detect when the medical device 120 completes a therapy session based on accumulation of data in the memory 106. For example, when the medical device 120 completes a therapy session as indicated by the data accumulated, the scheduler program 114 may control a transfer of any accumulated data associated with the newly completed therapy session to the remote server 130.

### 3.1.5 Flow Diagram

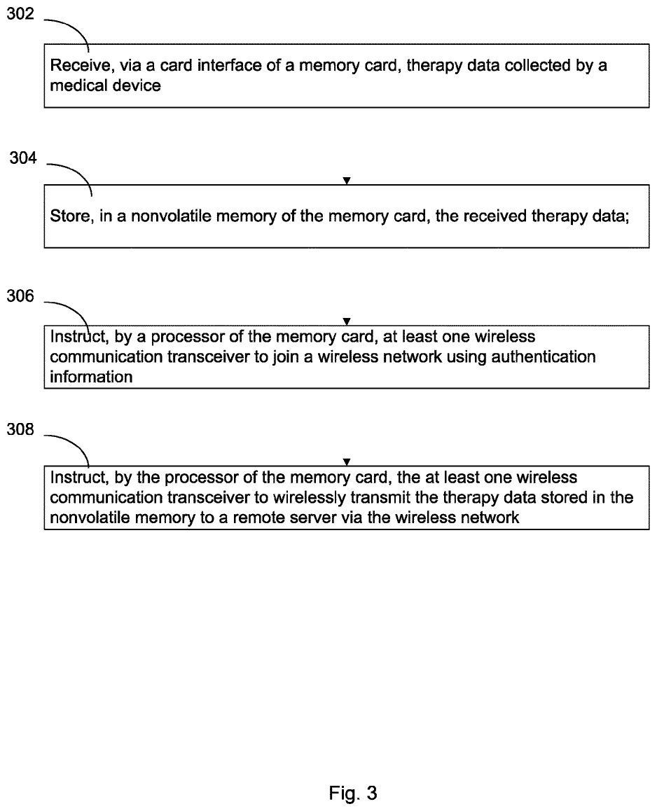

FIG. 3 illustrates a flow diagram with an example method for transmitting data to the remote server 130. At 302, the card interface 104 of the memory card 100 may receive data provided by the medical device 120. At 304, the non-volatile memory 107 may store the received data. At 306, the processor(s) 102 may instruct at least one wireless communication transceiver 110, such as the transceiver 112, to join a wireless network using authentication information. At 308, the processor(s) 102 may instruct the at least one wireless communication transceiver 110 to wirelessly transmit the data stored in the non-volatile memory 107 to the remote server 130 via the wireless network.

In one implementation, the memory card 100 may receive the authentication information from the medical device 120 via the card interface 104. In another implementation, the authentication information may be wirelessly received by the at least one wireless communication transceiver 110 from the wireless device 140.

The processor(s) 102 may instruct the at least one wireless communication transceiver 110 to access the remote server 130 using credential information. In some implementations, the credential information may be received from the medical device 120 via the card interface 104. In some implementations, the credential information may be wirelessly received from the wireless device 140. In some implementations, the credential information may be retrieved from a firmware of the memory card 100.

Although the aforementioned communications generally describe transmissions of data from the memory card 100 to the remote server 130, in some versions, the memory card 100 may be implemented to retrieve data (e.g., download or pull communication) from the remote server 130 or a related server of the system, or receive data from such a server (e.g., a push communication). Such received or retrieved data may be data useful for operation of the medical device 120. For example, the memory card 100 may obtain settings or control parameter data (e.g., an of flow rate settings, pressure settings, a software update etc.) from the remote server 130 for controlling operation of the medical device. The medical device 120 may then access the memory card 100 to obtain such data for control of the operations of the medical device 120. Similarly, the memory card 100 may obtain message data (e.g., a warning, use instructions or other information) from the remote server 130. The medical device 120 may then access the memory card 100 to obtain such message data and display the message data on a display of the medical device 120.

### 3.2 Barcode

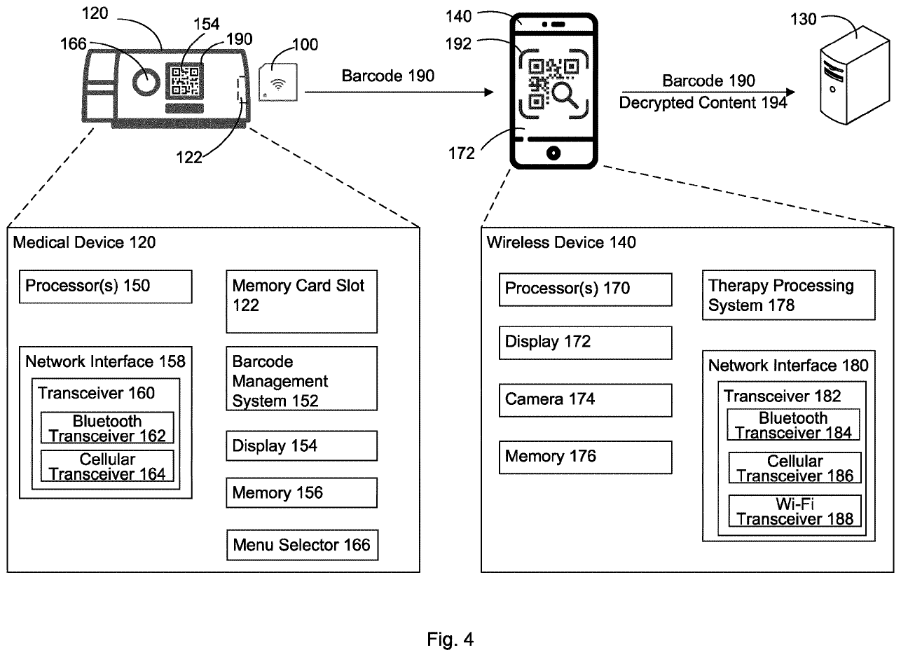

Another aspect of the present technology relates to exporting data from the medical device 120 via barcodes which are scannable by the wireless device 140 for transmission to the remote server 130. Such implementation may be considered in relation to FIGS. 4-7.

Referring to FIG. 4, according to one aspect of the present technology, the medical device 120 may generate and display a barcode 190 in its display 154. The barcode 190 may be unidimensional or may be multi-dimensional, such as a two-dimensional barcode, such as a quick response (QR) code. The barcode 190 may encode the following content: information of the medical device 120 or therapy data related to one or more treatment sessions performed with the medical device 120. The wireless device 140 may take a snapshot 192 of the barcode 190 (e.g., with an image sensor or camera), and transfer the barcode image or its decoded content 194, to the remote server 130. Alternatively, the wireless device 140 may decode the barcode 190 to obtain information of the medical device 120 and/or the therapy data. The wireless device 140 may encrypt information of the medical device 120 and/or the therapy data, and transfer such encrypted information of the medical device 120 and/or encrypted therapy data to the remote server 130.

### 3.2.1 Medical Device

As shown in FIG. 4, the medical device 120 may have one or more processors 150, the memory card slot 122 for removably receiving the memory card 100, a barcode management system 152 for generating the barcode 190, a display 154 for displaying the barcode 190, memory 156, a network interface 158 and a selector 166, such as an input device 4220 or control element of a graphic user interface. The network interface 158 may have one or more transceivers 160, such as a Bluetooth transceiver 162 and a cellular transceiver 164. The selector 166 may, for example, take the form of a knob, which may be manipulated by the user to navigate menus shown in the display 154.

In one example, the barcode management system 152 of the medical device 120 may be implemented with processor control instructions for operation of the processors 150. The barcode management system 152 may generate a first barcode encoding device information of the medical device 120. The first barcode may encode a device identifier and/or any one or more device settings of the medical device 120. The first barcode may be further configured to encode an encryption key. The encryption key may be later used by the wireless device 140 to perform encryption on the device identifier and/or device setting(s) of the medical device 120. The device identifier may be unique. An example of the device identifier may be a serial number of the medical device 120. In some implementations, the device identifier may be omitted or may be a unique identifier other than the serial number such as a patient identifier. The device settings may, for example, include any one or more of the following: therapy mode, maximum pressure, minimum pressure, and EPR pressure, among other possibilities. The barcode management system 152 of the medical device 120 may also generate a second barcode and/or subsequent barcode(s) encoding therapy data related to one or more treatment sessions performed by the medical device 120. In one example, the therapy data may be stored in the medical device 120 and/or the memory card 100 for a predetermined period of time, such as 180 days. In such a case, the bar code may encode a summary of the therapy data for that period of time.

In one example, at the end of a treatment session, the barcode management system 152 may generate a barcode with summarized or compressed therapy data associated with the treatment session. In one embodiment, the barcode management system 152 may also encrypt the data for inclusion in the bar code. Optionally, the user may manipulate the selector 166 to select a menu to display the barcode.

Alternatively, or additionally, the barcode management system 152 may periodically display such a bar code at predetermined times in a more automatic fashion such as at the conclusion of a therapy session or at the conclusion of a plurality of therapy sessions. Thus, at the end of the treatment session(s), after the barcode management system 152 automatically generates the barcode, the barcode may be automatically displayed on the display 154. As a result, the user may not need to go to any menu on the medical device 120 to search for the barcode.

Each time after the medical device 120 displays a barcode, the medical device 120 may track and detect whether the barcode has been scanned. For example, each time after the wireless device 140 scans the barcode displayed on the medical device 120, the medical device 120 may display a message through the display 154 informing the user that the barcode has been successfully scanned. Such tracking may be achieved with an application of the wireless device 140, such as the therapy processing system 178, that communicates a success message with the medical device 120, such as via a wireless link to the medical device 120, upon successful completion of the scanning.

### 3.2.2 Wireless Device

The wireless device 140 may include one or more processors 170, a display 172, a camera 174, memory 176, a therapy processing system 178 for processing the barcode 190, and a network interface 180. The network interface 180 may have one or more wireless transceivers 182, such as a Bluetooth transceiver 184, a cellular transceiver 186, and a Wi-Fi transceiver 188. The display 172 may be a monitor having a screen or any other electrical device that is operable to display information (e.g., text, imagery and/or other graphical elements). In addition, the wireless device 140 may include all of the components normally used in connection with a computing device such as a user interface subsystem. The user interface subsystem may include one or more user input devices (e.g., a mouse, keyboard, touch screen and/or microphone) for receiving input from the user, and output devices such as speaker(s).

The therapy processing system 178, which may be implemented with processor control instructions for operation of the processors 170, may prompt the user to scan or take a snapshot of the barcode 190 by using the camera 174. The therapy processing system 178 may generate prompts to the user on a regular basis. For instance, the therapy processing system 178 may remind the user on a daily basis to scan barcodes. Optionally, the therapy processing system 178 may also communicate one or more messages to the medical device 120, such as via a wireless link with the medical device, to prompt the bar code management system 152 to generate and display a bar code and/or identify when scanning has been successfully completed. In one example, the therapy processing system 178 may prompt the user to perform scan after detecting that the user has not scanned a barcode for a prolonged period of time. To perform a scan, the user may hold the wireless device 140 over the display 154 of the medical device 120 to scan the barcode.

In some implementations, the therapy processing system 178 may be configured to decode any scanned barcode to obtain its decoded content. The therapy processing system 178 may be configured to generate a summary of the decoded content. Based on or using the decoded content 194 or the summary of the decoded content, the therapy processing system 178 may display information related to the user's treatment session(s) to the user through the display 172.

The wireless device 140 may communicate, such as with the remote server 130 or medical device 120, via any of the following transceivers 182: the Bluetooth transceiver 184, the cellular transceiver 186 and the Wi-Fi transceiver.

### 3.2.3 Remote Server

The remote server 130 may store therapy data associated with treatment sessions of a plurality of users. Each user may have a user account at the remote server 130. Each user account may store historical therapy data obtained from the user's treatment sessions, so that the remote server 130 can track treatment progress of each individual user. Each user account may also store device information of the medical device used by the user.

The remote server 130 may receive from the therapy processing system 178 of the wireless device 140 any one of the following: an actual picture (i.e., image data) of the barcode 190, content of the barcode, decoded content 194 of the barcode, or a summary of the content or decoded content. In the event that the remote server 130 receives the barcode, the remote server 130 may perform decoding.

The remote server 130 may provide therapy support to the user through the therapy processing system 178 of the wireless device 140. For example, if the therapy processing system 178 does not perform decoding, the remote server 130 may decode the barcode to obtain decoded content. The remote server 130 may send the decoded content or a summary of the decoded content to the therapy processing system 178 for display to the user.

In implementations, instead of relying on the therapy processing system 178 of the wireless device 140, the remote server 130 may communicate with the user via a social media platform or a third-party application, such as WhatsApp. The remote server 130 may send a message to the user through the social media platform or third-party application, requesting the user to capture an image of the barcode or capture an image of the display 154 of the medical device 120 showing therapy data, such as in plain text, and request the user to transmit the captured image to the remote server 130 via the social media platform or the third-party application. The user's phone number or the user's social media identification may be used for verification purposes.

### 3.2.4 Initial Setup

When the user receives the medical device 120 for the first time, the user needs to register the medical device 120 with the remote server. For example, the user may download and install the therapy processing system 178 onto the user's wireless device 140, and create a user account at the remote server 130 through the therapy processing system 178. The therapy processing system 178 may provide the functionality described herein as well as a graphic user interface for such functionality.

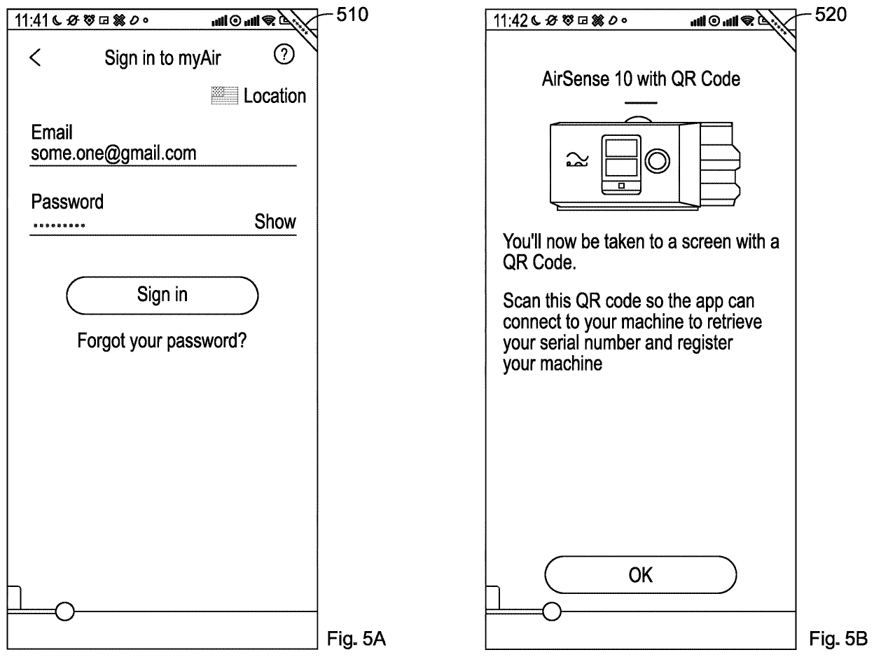

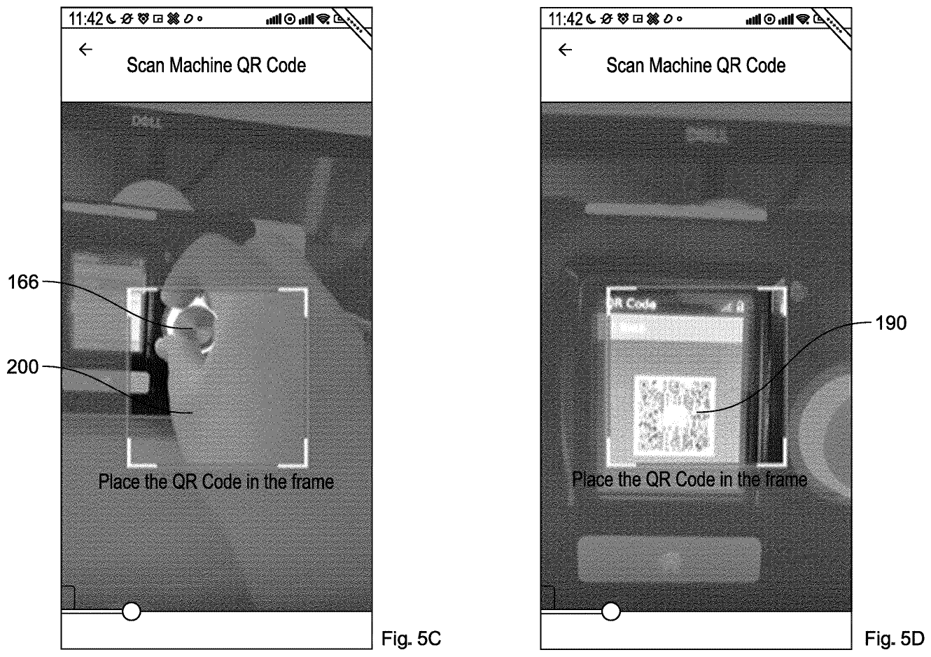

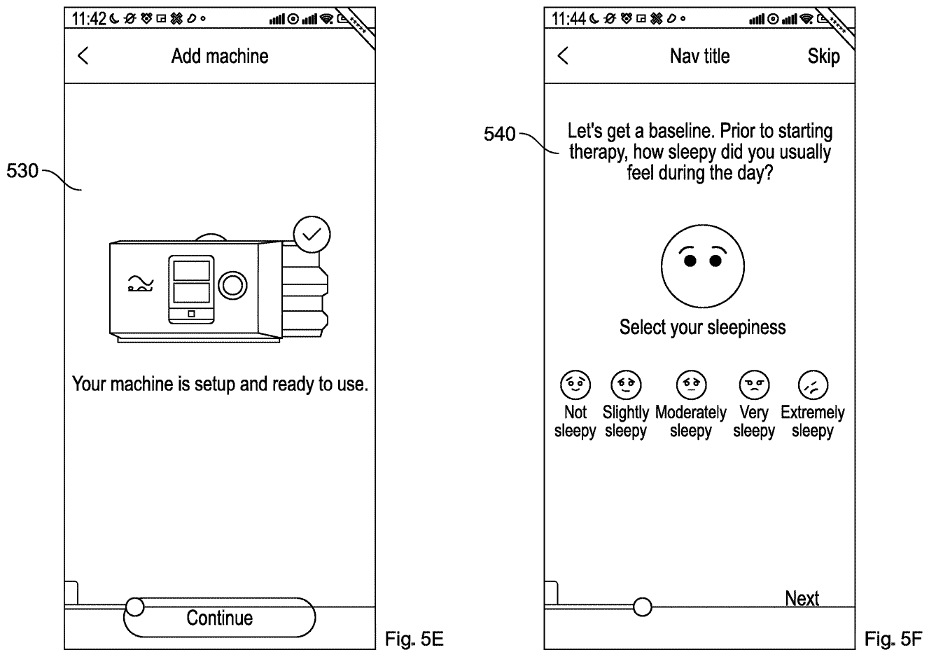

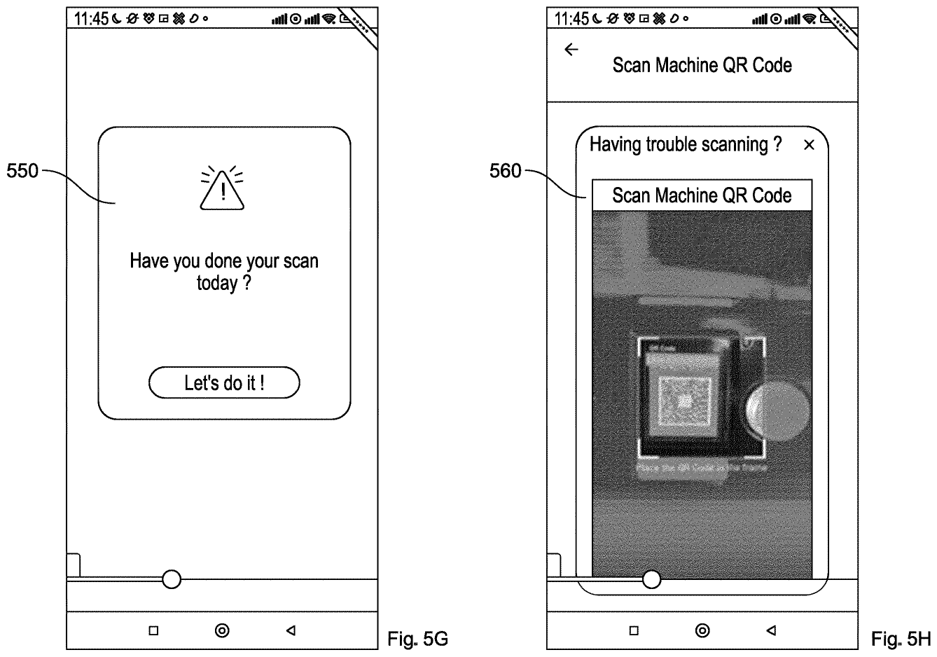

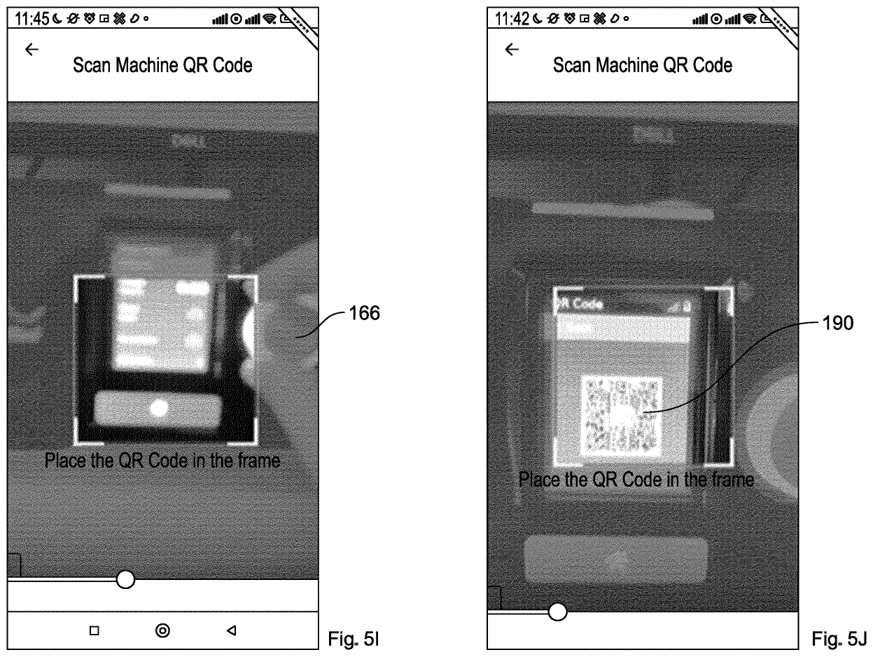

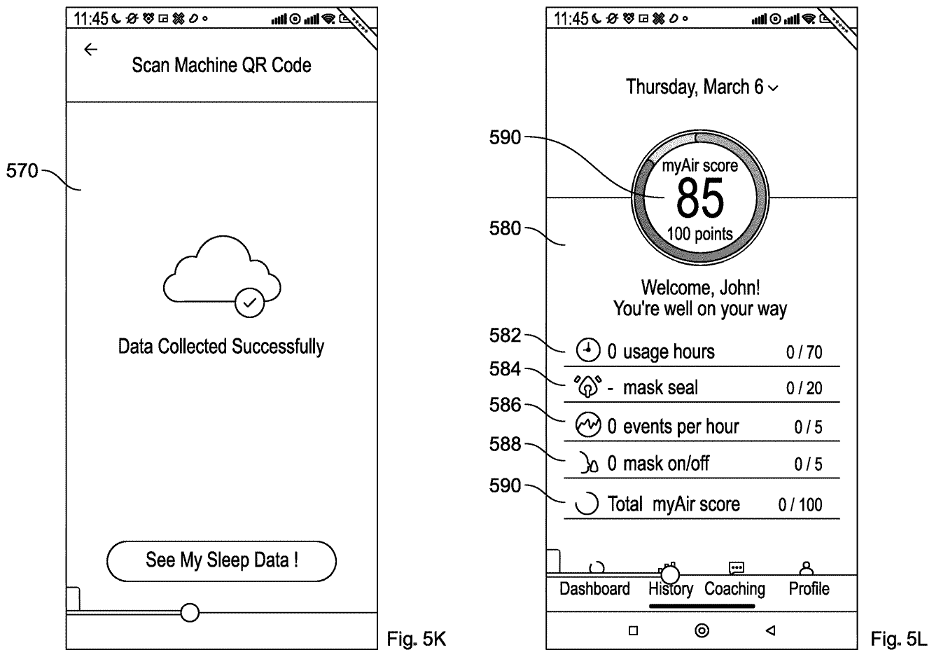

In one such example, as shown in FIG. 5A, the therapy processing system 178 (e.g., referred to as my Air in FIG. 5A) may display an initial page 510, requesting the user to sign in. Such sign in information may serve as credentials for communicating with the remote server 130. Next, as shown in FIG. 5B, the therapy processing system 178 may display a page 520 requesting the user to scan a barcode from the medical device 120. Thereafter, as shown in FIG. 5C, the user 200 may then use the selector 166 of the medical device 120 to select an option or menu to display a first barcode that encodes device information of the medical device 120.

Referring to FIG. 5D, the user may scan the first barcode 190 displayed on the medical device 120. Once the first barcode is scanned, the therapy processing system 178 may send the first barcode or its decoded content to the remote server 130, so as to register the medical device 120 with the remote server 130. By doing so, the remote server 130 may store device information of the medical device under the user account.

In one embodiment, the first barcode may encode device information of the medical device 120. For example, the first barcode may encode a device identifier and/or any one or more device settings of the medical device. In addition, the first barcode may encode an encryption key. Once the first barcode is scanned by the wireless device 140, the therapy processing system 178 may decode the first barcode to retrieve device information of the medical device 120 such as the device identifier and/or device setting(s), and may also retrieve the encryption key. The therapy processing system 178 may store the encryption key in the memory 176. The therapy processing system 178 may encrypt the device information including the device identifier and/or device setting(s) by using the encryption key, and send the encrypted device information to the remote server 130. The remote server 130 may perform decryption to obtain the device information so as to register the medical device 120 with the remote server 130. By doing so, the remote server 130 may store the device information of the medical device under the user account.

As shown in FIG. 5E, after completing the above initial setup, the therapy processing system 178 may display a page 530 showing that the medical device 120 is ready to use. The therapy processing 178 may then display a subsequent page 540 to get a baseline of the user's status, such as prompting the user to answer how sleepy the user usually feels during the day as shown in FIG. 5F.

In some implementations, the user may register the medical device 120 with the remote server without relying on the first barcode. Thus, the therapy processing system 178 may obtain device information of the medical device 120 without relying on displaying a barcode. For example, device information of the medical device 120, such as the serial number, may be acquired by capturing an image of a label on the medical device 120. Alternatively, such device information may be entered by the user into the therapy processing system 178, through keyboard input or audio input. Once the therapy processing system 178 obtains the device information, it may send the device information to the remote server 130 to complete registration of the medical device 120.

### 3.2.5 Routine Scan

Once the medical device 120 is registered at the remote server 130, the user may routinely or periodically upload the therapy data captured by the medical device 120 to the remote server 130 after each treatment session, by using the wireless device 140 to scan the second or subsequent barcode displayed by the medical device 120.

In this regard, the therapy processing system 178 may regularly or periodically prompt the user to scan the barcode. For example, as shown in FIG. 5G, the therapy processing system 178 may display a prompt 550 to the user requesting the user to perform scan. If there is no scan received from user after a period of time, such as 6 seconds, the therapy processing system 178 may display a page 560 as shown in FIG. 5H prompting the user to attend a tutorial. As shown in FIG. 5I, the user may manipulate the selector 166 to select a menu to display a barcode that encodes, and optionally, encrypts therapy data of one or more treatment sessions. Therapy data may include, but not limited to, one or more respiratory measurements of the user as collected by the medical device 120, usage data of the medical device 120, and one or more device settings of the medical device 120 used in the user's treatment session(s). The respiratory measurements may include how many respiratory events a user has experienced, such as number of respiratory events per hour. The usage data may include how much time the medical device has been used such as usage hours, and how much time a mask has been worn by the user during one or more treatment sessions, number of times that the mask is on and off the user, and efficiency of mask seal, among others.

The device settings may include one or more of the following parameters of the medical device: therapy mode, maximum pressure, minimum pressure, and expiratory pressure relief (EPR) pressure, among other possibilities. By way of example, in one treatment setting, the maximum pressure may be 20 cmH₂O. The minimum pressure may be 4 cmH₂O. The therapy mode may be AutoSet, indicating, for example, a pressure range of 4 cmH₂O to 20 cmH₂O (4-20 hPa). In such an AutoSet mode, the device may then modify the delivered therapy pressure, within the range, depending on detection of respiratory events (e.g., a pressure increase for detected flow limitation or obstructive apnea) or an absence of detection of such events (e.g., a pressure decrease). The EPR, if enabled, may be selected to provide one of three levels, where level 1 may indicate 1.0 cmH₂O (1 hPa), level 2 may indicate 2.0 cmH₂O (2 hPa), and level 3 may indicate 3.0 cmH₂O (3 hPa). The EPR may then operate the device to provide a reduction of the therapy pressure during expiration by the amount of the EPR setting. Such a reduction in pressure delivered will not typically drop below a predetermined threshold, such as 4 cmH₂O (4 hPa).

As shown in FIG. 5J, the wireless device 140 may scan the barcode that appears on the display 154 of the medical device 120. After a successful scan, as shown in FIG. 5K, the therapy processing system 178 may display a page 570 informing the user that data has been successfully collected.

In one embodiment, the second barcode and/or any subsequent barcode may encode the therapy data of one or more treatment sessions. During routine scans, after the second barcode and/or subsequent barcode(s) is scanned, the therapy processing system 178 may decode the second barcode and/or subsequent barcode(s) to retrieve the therapy data. The therapy processing system 178 may encrypt the therapy data and/or a summary of the therapy data, by using the encryption key retrieved from the first barcode. The therapy processing system 178 may send encrypted therapy data or encrypted summary of the therapy data to the remote server 130. The remote server 130 may perform decryption to obtain the therapy data and/or summary of the therapy data.

In another embodiment, the therapy processing system 178 may decode the content of the barcode, and may decrypt the barcode to obtain decrypted content. The therapy processing system 178 may generate a summary of the decoded/decrypted content. The therapy processing system 178 may automatically transfer to the remote server 130 one or more of the following: the barcode, its decoded/decrypted content, or the summary of the decoded/decrypted content.

In yet another embodiment, the therapy processing system 178 may refrain from decrypting or decoding the barcode and instead merely send the barcode directly to the remote server 130. The remote server 130 in turn may perform decoding/decryption, and send the decoded/decrypted content or a summary of the decoded/decrypted content to the therapy processing system 178 for display to the user.

The therapy processing system 178 may display a page 580 showing the decoded/decrypted content or a summary of the decoded/decrypted content as shown in FIG. 5L. In one example, the therapy processing system 178 may display usage hours 582, efficiency of mask seal 584, respiratory events per hour 586, number of times that the mask is on and off the user 588 and a total sleep score 590 or a therapy quality indicator based on such data.

### 3.2.6 Flow Diagrams

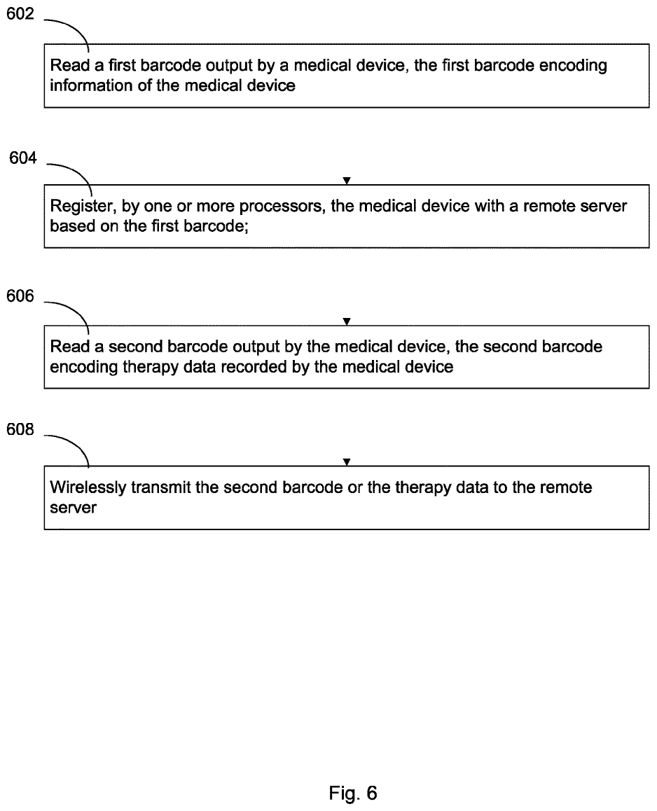

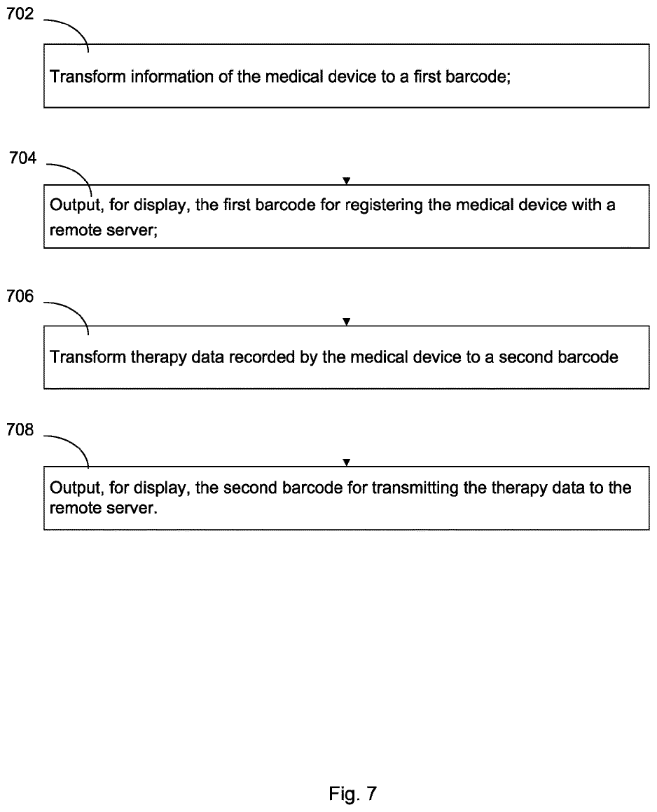

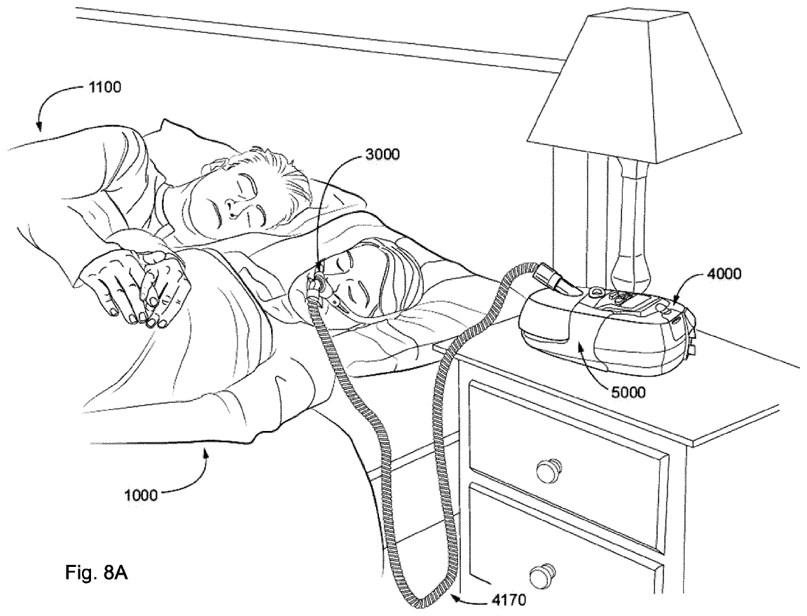

Methodologies of the present technology may be further considered in relation to the flow charts of FIGS. 6 and 7. FIG. 6 illustrates an example data reading process performed by the therapy processing system 178 of the wireless device 140. At 602, one or more processors 170 may read a first barcode output by the medical device 120. The medical device 120 may be a respiratory pressure therapy device. The first barcode may encode information of the medical device. At 604, the one or more processors 170 may register the medical device with a remote server 130 based on the first barcode. At 606, the one or more processors 170 may read a second barcode output by the medical device. The second barcode may encode therapy data recorded by the medical device. At 608, the second barcode or the therapy data may be wirelessly transmitted to the remote server.

In one example, the processor(s) 170 may decode the first barcode to obtain the information of the medical device. The wireless transceiver 182 may transmit the decoded information of the medical device to the remote server 130.

In another example, the first barcode may be further configured to encode an encryption key. The processor(s) 170 may decode the first barcode to obtain the information of the medical device and the encryption key. The processor(s) 170 may encrypt the information of the medical device by using the encryption key. The wireless transceiver 182 may transmit the encrypted information of the medical device to the remote server.

The information of the medical device may include at least one of the following: a device identifier and/or one or more device settings. The device settings may include one or more of the following parameters of the medical device: therapy mode, maximum pressure, minimum pressure, and expiratory pressure relief (EPR) pressure, among other possibilities. In one example, each of the first barcode and the second barcode is a two-dimensional code.

In one example, the processor(s) 170 may decode the second barcode to obtain the therapy data recorded by the medical device. The wireless transceiver 182 may wirelessly transmit the therapy data to the remote server after the second barcode is decoded.

In another example, the processor(s) 170 may decode the second barcode to obtain the therapy data recorded by the medical device. The processor(s) 170 may encrypt the therapy data by using the encryption key obtained from the first barcode. The wireless transceiver 182 may wirelessly transmit the encrypted therapy data to the remote server 130.

A prompt may be displayed to the user to scan the second barcode according to a predetermined schedule. The processor(s) 170 may determine whether the second barcode has been scanned according to the predetermined schedule. When the second barcode has not been read according to the predetermined schedule, a prompt may be displayed to the user to scan the second barcode.

The therapy data may include one or more of the following: one or more respiratory measurements of the user as collected by the medical device 120, usage data of the medical device 120, and one or more device settings of the medical device 120 when treating the user. Therapy data may also include data measurements of any one or more of the sensors described herein or other determined information or parameters, such as any from the pre-processing module 4310 and/or the therapy engine module 4320.

FIG. 7 illustrates a process for reporting therapy data of the medical device 120, to the remote server 130. At 702, one or more processor(s) 150 may transform information of the medical device to a first barcode. At 704, the processor(s) 150 may output for display the first barcode for registering the medical device with the remote server 130. At 706, the processor(s) 150 may transform therapy data recorded by the medical device to a second barcode. At 708, the processor(s) 150 may output, for display, the second barcode for transmitting the therapy data to the remote server 130.

In one example, the information of the medical device transformed to the first barcode may include one or more of the following: a serial number, one or more device settings, and an encryption key of the medical device.

### 3.3 Technical Advantages

The present technology disclosed herein presents long-term, cost effective and scalable solutions for providing data, such as high resolution data, to a remote server.

With the wireless memory card 100 and barcode solutions disclosed herein, therapy data can be exported to a remote server from anywhere that has network access, such as at the comfort of the user's home. The user no longer needs to visit a clinician's office to export therapy data.

The wireless memory card 100 disclosed herein enables high resolution data transmission via a transceiver (e.g., Wi-Fi) that offers a larger bandwidth than a cellular modem. As a result, the wireless memory card 100 can send higher resolution data than existing respiratory therapy devices that rely on cellular modems are able to handle. Further, due to its wireless transfer capability, the memory card 100 can perform data export to any remote server while being mounted on the medical device 120, eliminating any need to unplug the memory card from the medical device and plugging the memory card to another computing device, such as at a remote clinician's office.

### 3.4 Example Memories and Processors

The memory 106, 156 and 176 described herein may be databases that store information accessible by the processor(s) 102, 150 and 170, respectively. For example, the memory 106 of the memory card 100 may store instructions (e.g., processor control instructions) and data associated with the scheduler program 114 that may be executed or otherwise used by the processor(s) 102. The memory 156 of the medical device 120 may store instructions and data associated with the barcode management system 152 that may be executed or otherwise used by the processor(s) 150. The memory 176 of the wireless device 140 may store instructions and data associated with the therapy processing system 178 that may be executed or otherwise used by the processor(s) 170. The memory 106, 156 and 176 may be of any type capable of storing information accessible by the processor(s), including a computing device-readable medium. The memory may be a non-transitory medium such as a hard-drive, memory card, optical disk, solid-state, etc. The memory may include different combinations of the foregoing, whereby different portions of the instructions and data are stored on different types of media. The instructions may be any set of instructions to be executed directly (such as machine code) or indirectly (such as scripts) by the processor(s). For example, the instructions may be stored as computing device code on the computing device-readable medium. In that regard, the terms “instructions”, “modules” and “programs” may be used interchangeably herein. The instructions may be stored in object code format for direct processing by the processor, or in any other computing device language including scripts or collections of independent source code modules that are interpreted on demand or compiled in advance.

The processors 102, 150 and 170 may be any conventional processors, such as commercially available GPUs, CPUs, TPUs, etc. Alternatively, each processor may be a dedicated device such as an ASIC or other hardware-based processor. Although FIGS. 1A to 2D and FIG. 4 functionally illustrate the processors, memory as being within the same block, such devices may actually include multiple processors, computing devices, or memories that may or may not be stored within the same physical housing. Similarly, the memory may be a hard drive or other storage media located in a housing different from that of the processor(s), for instance in a cloud computing system. Accordingly, references to a processor or computing device will be understood to include references to a collection of processors or computing devices or memories that may or may not operate in parallel. The processors 102, 150 and 170 may respectively access the memory 106, 156 and 176 via a network.

### 4.1 Optional Example Treatment Systems

An example embodiment of the medical device 120 is discussed in sections 4.1 to 4.5.

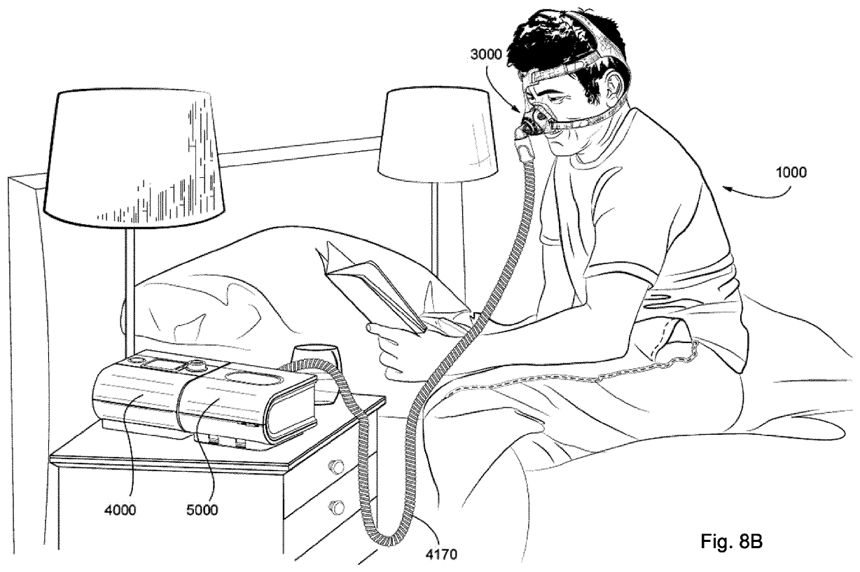

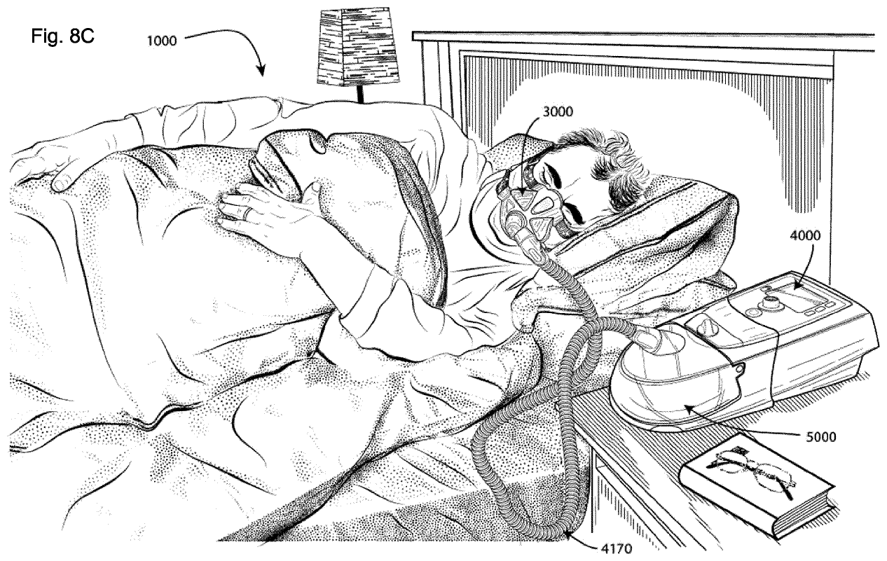

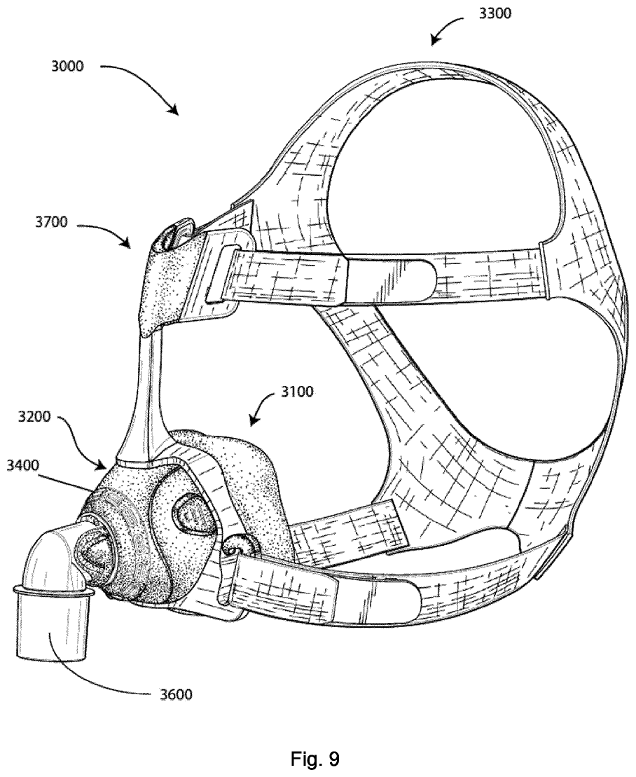

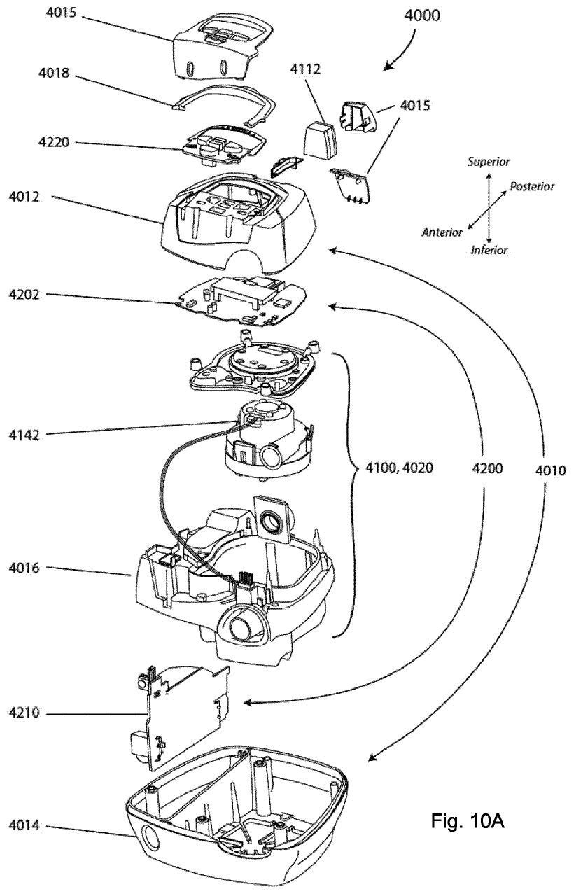

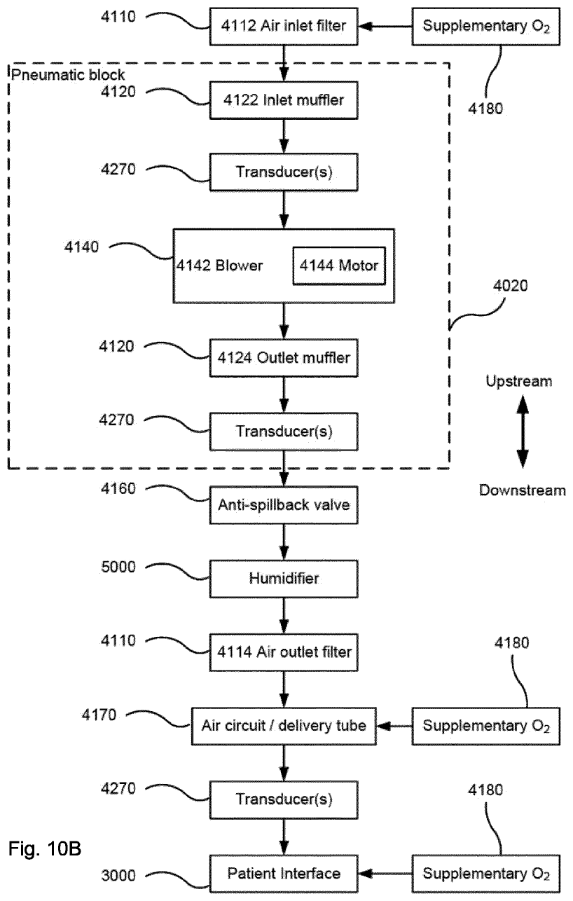

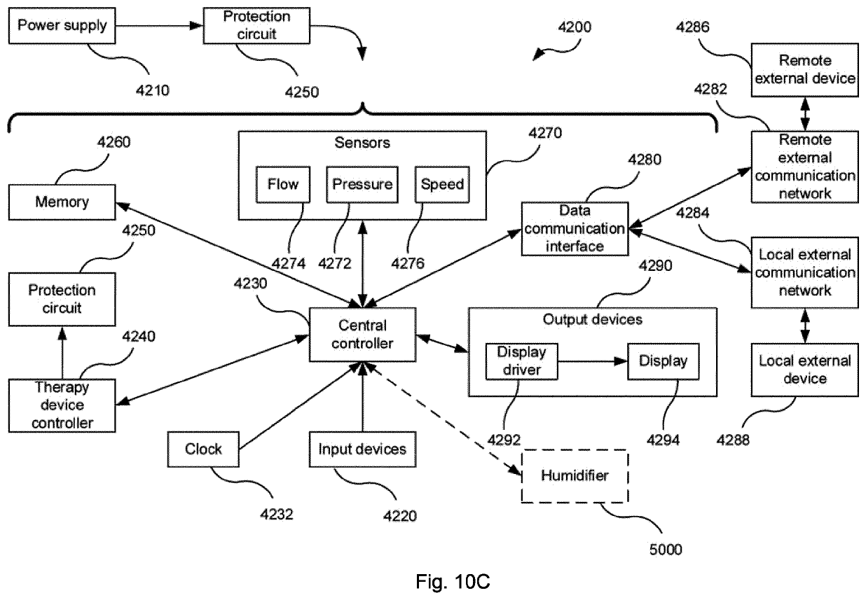

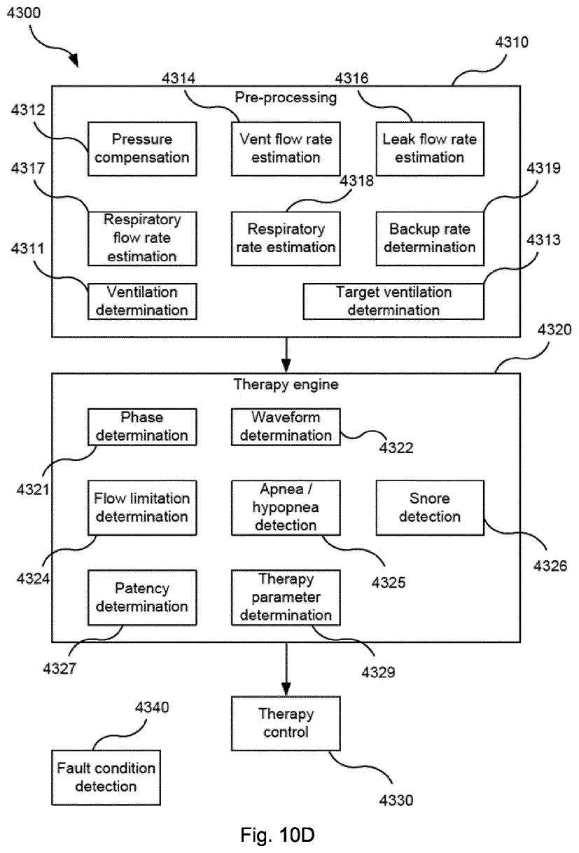

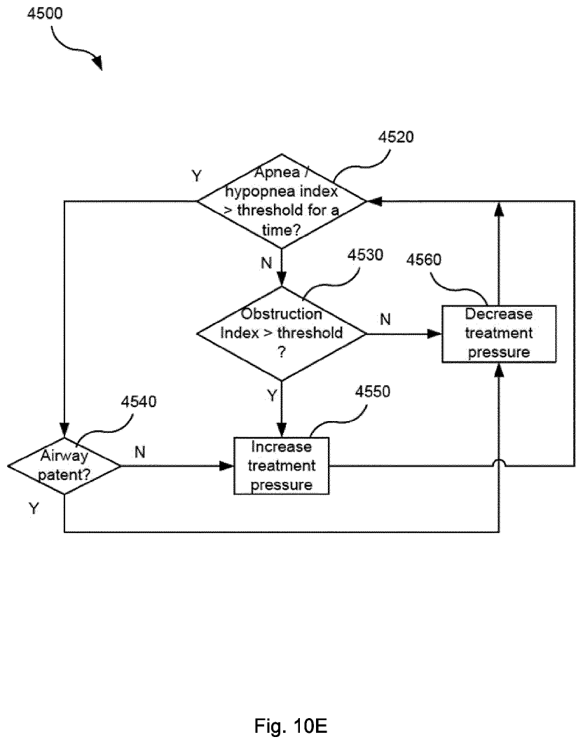

In one form, the medical device 120 may treat and/or monitor a respiratory disorder. The medical device 120 may be a respiratory therapy device (RT) such as an RPT device 4000 for supplying a flow of pressurised air to the patient 1000 via an air circuit 4170 leading to a patient interface 3000. The flow of air may be pressure-controlled (for respiratory pressure therapies) or flow-controlled (for flow therapies such as high flow therapy HFT). Thus, RPT devices may also be configured to act as flow therapy devices, such as when using a patient interface that does not use a seal that seals with the patient's respiratory system. In the following description, the RT or RPT device may be considered in reference to FIGS. 8A-11.

### 4.2 Patient Interface

As shown in FIG. 9, a non-invasive patient interface 3000 in accordance with one aspect of the present technology may optionally comprise any of the following functional aspects: a seal-forming structure 3100, a plenum chamber 3200, a positioning and stabilising structure 3300, a vent 3400, a connection port 3600 for connection to air circuit 4170, and a forehead support 3700. In some forms a functional aspect may be provided by one or more physical components. In some forms, one physical component may provide one or more functional aspects. In use the seal-forming structure 3100 is arranged to surround an entrance to an airway of the patient so as to facilitate the supply of pressurised air to the airway.

### 4.3 RPT Device

An RPT device 4000 in accordance with one aspect of the present technology comprises mechanical and pneumatic components 4100, electrical components 4200 and is programmed to execute one or more algorithms 4300. The RPT device 4000 may have an external housing 4010 formed in two parts, an upper portion 4012 and a lower portion 4014. In one form, the external housing 4010 may include one or more panel(s) 4015. The RPT device 4000 may comprise a chassis 4016 that supports one or more internal components of the RPT device 4000. The RPT device 4000 may include a handle 4018.

The pneumatic path of the RPT device 4000 may comprise one or more air path items, e.g., an inlet air filter 4112, an inlet muffler 4122, a pressure generator 4140 capable of supplying pressurised air (e.g., a blower 4142), an outlet muffler 4124, and one or more transducers 4270, such as pressure sensors 4272 and flow rate sensors 4274.

One or more of the air path items may be located within a removable unitary structure which will be referred to as a pneumatic block 4020. The pneumatic block 4020 may be located within the external housing 4010. In one form a pneumatic block 4020 is supported by, or formed as part of the chassis 4016.

The RPT device 4000 may have an electrical power supply 4210, one or more input devices 4220, a central controller 4230, a therapy device controller 4240, a pressure generator 4140, one or more protection circuits 4250, memory 4260, transducers 4270, data communication interface 4280 and one or more output devices 4290. Electrical components 4200 may be mounted on a single Printed Circuit Board Assembly (PCBA) 4202. In an alternative form, the RPT device 4000 may include more than one PCBA 4202.

### 4.3.1 RPT Device Mechanical & Pneumatic Components

An RPT device 4000 may comprise one or more of the following components in an integral unit. In an alternative form, one or more of the following components may be located as respective separate units.

### 4.3.1.1 Air Filter(s)

An RPT device 4000 in accordance with one form of the present technology may include an air filter 4110, or a plurality of air filters 4110.

In one form, an air inlet filter 4112 is located at the beginning of the pneumatic path upstream of a pressure generator 4140.

In one form, an air outlet filter 4114, for example an antibacterial filter, is located between an outlet of the pneumatic block 4020 and a patient interface 3000.

### 4.3.1.2 Muffler(s)

An RPT device 4000 in accordance with one form of the present technology may include a muffler 4120, or a plurality of mufflers 4120.

In one form of the present technology, an inlet muffler 4122 is located in the pneumatic path upstream of a pressure generator 4140.

In one form of the present technology, an outlet muffler 4124 is located in the pneumatic path between the pressure generator 4140 and a patient interface 3000.

### 4.3.1.3 Pressure Generator

In one form of the present technology, a pressure generator 4140 for supplying pressurised air is a controllable blower 4142. For example, the blower 4142 may include a brushless DC motor 4144 with one or more impellers housed in a volute. The pressure generator 4140 may be capable of generating a supply or flow of air, for example at about 120 litres/minute, at a positive pressure in a range from about 4 cmH₂O to about 20 cmH₂O, or in other forms up to about 30 cmH₂O.

The pressure generator 4140 is under the control of the therapy device controller 4240.

In other forms, a pressure generator 4140 may be a piston-driven pump, a pressure regulator connected to a high pressure source (e.g., compressed air reservoir), or a bellows.

### 4.3.1.4 Transducer(s)

Transducers may be internal of the RPT device, or external of the RPT device. External transducers may be located for example on or form part of the air circuit, e.g., the patient interface. External transducers may be in the form of non-contact sensors such as a Doppler radar movement sensor that transmit or transfer data to the RPT device.

In one form of the present technology, one or more transducers 4270 are located upstream and/or downstream of the pressure generator 4140. The one or more transducers 4270 are constructed and arranged to generate data representing respective properties of the air flow, such as a flow rate, a pressure or a temperature, at that point in the pneumatic path.

In one form of the present technology, one or more transducers 4270 are located proximate to the patient interface 3000.

In one form, a signal from a transducer 4270 may be filtered, such as by low-pass, high-pass or band-pass filtering.

### 4.3.1.5 Anti-Spill Back Valve

In one form of the present technology, an anti-spill back valve 4160 is located between the humidifier 5000 and the pneumatic block 4020. The anti-spill back valve is constructed and arranged to reduce the risk that water will flow upstream from the humidifier 5000, for example to the motor 4144.

### 4.3.1.6 Air Circuit

An air circuit 4170 in accordance with one aspect of the present technology is a conduit or tube constructed and arranged to allow, in use, a flow of air to travel between two components such as the pneumatic block 4020 and the patient interface 3000.

### 4.3.1.7 Oxygen Delivery

In one form of the present technology, supplemental oxygen 4180 is delivered to one or more points in the pneumatic path, such as upstream of the pneumatic block 4020, to the air circuit 4170 and/or to the patient interface 3000.

### 4.3.2 RPT Device Electrical Components

### 4.3.2.1 Power Supply

In one form of the present technology power supply 4210 is internal of the external housing 4010 of the RPT device 4000. In another form of the present technology, power supply 4210 is external of the external housing 4010 of the RPT device 4000.

In one form of the present technology power supply 4210 provides electrical power to the RPT device 4000 only. In another form of the present technology, power supply 4210 provides electrical power to both RPT device 4000 and humidifier 5000.

### 4.3.2.2 Input Devices

In one form of the present technology, an RPT device 4000 includes one or more input devices 4220 in the form of buttons, switches or dials to allow a person to interact with the device. The buttons, switches or dials may be physical devices, or software devices accessible via a touch screen. The buttons, switches or dials may, in one form, be physically connected to the external housing 4010, or may, in another form, be in wireless communication with a receiver that is in electrical connection to the central controller 4230.

In one form the input device 4220 may be constructed and arranged to allow a person to select a value and/or a menu option.

### 4.3.2.3 Central Controller

In one form of the present technology, the central controller 4230 is a processor suitable to control an RPT device 4000 such as an x86 INTEL processor.

A central controller 4230 suitable to control an RPT device 4000 in accordance with another form of the present technology includes a processor based on ARM Cortex-M processor from ARM Holdings. For example, an STM32 series microcontroller from ST MICROELECTRONICS may be used.

Another central controller 4230 suitable to control an RPT device 4000 in accordance with a further alternative form of the present technology includes a member selected from the family ARM9-based 32-bit RISC CPUs. For example, an STR9 series microcontroller from ST MICROELECTRONICS may be used.

In certain alternative forms of the present technology, a 16-bit RISC CPU may be used as the central controller 4230 for the RPT device 4000. For example, a processor from the MSP430 family of microcontrollers, manufactured by TEXAS INSTRUMENTS, may be used.

In another form of the present technology, the central controller 4230 is a dedicated electronic circuit. In another form, the central controller 4230 is an application-specific integrated circuit (ASIC). In another form, the central controller 4230 comprises discrete electronic components.

The central controller 4230 is configured to receive input signal(s) from one or more transducers 4270, one or more input devices 4220, and the humidifier 5000.

The central controller 4230 is configured to provide output signal(s) to one or more of an output device 4290, a therapy device controller 4240, a data communication interface 4280, and the humidifier 5000.

In some forms of the present technology, the central controller 4230 is configured to implement the one or more methodologies described herein, such as the one or more algorithms 4300, expressed as computer programs stored in a non-transitory computer readable storage medium, such as memory 4260 or other memory described herein. In some forms of the present technology, as previously discussed, the central controller 4230 may be integrated with an RPT device 4000. However, in some forms of the present technology, some methodologies may be performed by a remotely located device or server such as the server previously mentioned. For example, the remotely located device or server may determine control settings for transfer to a ventilator or other RT device such as by detecting respiratory related events and distinguishing them by type by an analysis of stored data such as from any of the sensors described herein.

While the central controller 4230 may comprise a single controller interacting with various sensors 4270, data communications interface 4280, memory 4260, as well as other devices, the functions of controller 4230 may be distributed among more than one controller. Thus, the term “central” as used herein is not meant to limit the architecture to a single controller or processor that controls the other devices. For example, alternative architectures may include a distributed controller architecture involving more than one controller or processor, which may optionally be directly or indirectly in electronic (wired or wireless) communications with the previously described finger sensor or a server in communication with the finger sensor, such as for implementing any of the methodologies described herein. This may include, for example, a separate local (i.e., within RPT device 4000) or remotely located controller that perform some of the algorithms 4300, or even more than one local or remote memory that stores some of the algorithms. In addition, the algorithms when expressed as computer programs may comprise high level human readable code (e.g., C++, Visual Basic, other object oriented languages, etc.) or low/machine level instructions (Assembler, Verilog, etc.). Depending on the functionality of an algorithm(s), such code or instructions may be burnt in the controller, e.g., an ASIC or DSP, or be a run time executable ported to a DSP or general purpose processor that then becomes specifically programmed to perform the tasks required by the algorithm(s).

### 4.3.2.4 Clock

The RPT device 4000 may include a clock 4232 that is connected to the central controller 4230.

### 4.3.2.5 Therapy Device Controller

In one form of the present technology, therapy device controller 4240 is a therapy control module 4330 that forms part of the algorithms 4300 executed by the central controller 4230.

In one form of the present technology, therapy device controller 4240 is a dedicated motor control integrated circuit. For example, in one form a MC33035 brushless DC motor controller, manufactured by ONSEMI is used.

### 4.3.2.6 Protection Circuits

An RPT device 4000 in accordance with the present technology may comprise one or more protection circuits 4250.

One form of protection circuit 4250 in accordance with the present technology is an electrical protection circuit.

One form of protection circuit 4250 in accordance with the present technology is a temperature or pressure safety circuit.

### 4.3.2.7 Memory

In accordance with one form of the present technology the RPT device 4000 includes memory 4260, for example non-volatile memory. In some forms, memory 4260 may include battery powered static RAM. In some forms, memory 4260 may include volatile RAM.

Memory 4260 may be located on PCBA 4202. Memory 4260 may be in the form of EEPROM, or NAND flash.

Additionally or alternatively, RPT device 4000 includes a removable form of memory 4260, for example a memory card made in accordance with the Secure Digital (SD) standard.

In one form of the present technology, the memory 4260, such as any of the memories previously described, acts as a non-transitory computer readable storage medium on which is stored computer program instructions expressing the one or more methodologies described herein, such as the one or more algorithms 4300.

### 4.3.2.8 Transducers

Transducers may be internal of the device 4000, or external of the RPT device 4000. External transducers may be located for example on or form part of the air delivery circuit 4170, e.g., at the patient interface 3000. External transducers may be in the form of non-contact sensors such as a Doppler radar movement sensor that transmit or transfer data to the RPT device 4000.

### 4.3.2.8.1 Flow Rate

A flow rate transducer 4274 in accordance with the present technology may be based on a differential pressure transducer, for example, an SDP600 Series differential pressure transducer from SENSIRION. The differential pressure transducer is in fluid communication with the pneumatic circuit, with one of each of the pressure transducers connected to respective first and second points in a flow restricting element.

In one example, a signal representing total flow rate Qt from the flow transducer 4274 is received by the central controller 4230.

### 4.3.2.8.2 Pressure

A pressure transducer 4272 in accordance with the present technology is located in fluid communication with the pneumatic path. An example of a suitable pressure transducer 4272 is a sensor from the HONEYWELL ASDX series. An alternative suitable pressure transducer is a sensor from the NPA Series from GENERAL ELECTRIC.

In use, a signal from the pressure transducer 4272 is received by the central controller 4230. In one form, the signal from the pressure transducer 4272 is filtered prior to being received by the central controller 4230.

### 4.3.2.8.3 Motor Speed

In one form of the present technology a motor speed transducer 4276 is used to determine a rotational velocity of the motor 4144 and/or the blower 4142. A motor speed signal from the motor speed transducer 4276 may be provided to the therapy device controller 4240. The motor speed transducer 4276 may, for example, be a speed sensor, such as a Hall effect sensor.

### 4.3.2.9 Data Communication Systems

In one form of the present technology, a data communication interface 4280 is provided, and is connected to the central controller 4230. Data communication interface 4280 may be connectable to a remote external communication network 4282 and/or a local external communication network 4284. The remote external communication network 4282 may be connectable to a remote external device 4286. The local external communication network 4284 may be connectable to a local external device 4288.

In one form, data communication interface 4280 is part of the central controller 4230. In another form, data communication interface 4280 is separate from the central controller 4230, and may comprise an integrated circuit or a processor.

In one form, remote external communication network 4282 is the Internet. The data communication interface 4280 may use wired communication (e.g., via Ethernet, or optical fibre) or a wireless protocol (e.g., CDMA, GSM, LTE) to connect to the Internet.

In one form, local external communication network 4284 utilises one or more communication standards, such as Bluetooth, or a consumer infrared protocol and may optionally communicate with any of the sensors described herein.

In one form, remote external device 4286 is one or more computers, for example a cluster of networked computers and/or server as described herein. In one form, remote external device 4286 may be virtual computers, rather than physical computers. In either case, such a remote external device 4286 may be accessible to an appropriately authorised person such as a clinician.

The local external device 4288 may be a personal computer, mobile phone, tablet or remote control.

### 4.3.2.10 Output Devices Including Optional Display, Alarms

An output device 4290 in accordance with the present technology may take the form of one or more of a visual, audio and haptic unit. A visual display may be a Liquid Crystal Display (LCD) or Light Emitting Diode (LED) display.

### 4.3.2.10.1 Display Driver

A display driver 4292 receives as an input the characters, symbols, or images intended for display on the display 4294, and converts them to commands that cause the display 4294 to display those characters, symbols, or images.

### 4.3.2.10.2 Display

A display 4294 is configured to visually display characters, symbols, or images in response to commands received from the display driver 4292. For example, the display 4294 may be an eight-segment display, in which case the display driver 4292 converts each character or symbol, such as the figure “0”, to eight logical signals indicating whether the eight respective segments are to be activated to display a particular character or symbol.

### 4.3.3 RPT Device Algorithms

### 4.3.3.1 Pre-Processing Module

A pre-processing module 4310 in accordance with the present technology receives, as an input, raw data from a transducer 4270, for example a flow rate sensor 4274 or a pressure sensor 4272, and performs one or more process steps to calculate one or more output values that will be used as an input to another module, for example a therapy engine module 4320.

In one form of the present technology, the output values include the interface or mask pressure Pm, the respiratory flow rate Qr, and the leak flow rate Ql.

In various forms of the present technology, the pre-processing module 4310 comprises one or more of the following algorithms: pressure compensation 4312, vent flow rate estimation 4314, leak flow rate estimation 4316, respiratory flow rate estimation 4317, ventilation determination 4311, target ventilation determination 4313, respiratory rate estimation 4318, and backup rate determination 4319.

### 4.3.3.1.1 Pressure Compensation

In one form of the present technology, a pressure compensation algorithm 4312 receives as an input a signal indicative of the pressure in the pneumatic path proximal to an outlet of the pneumatic block 4020. The pressure compensation algorithm 4312 estimates the pressure drop in the air circuit 4170 and provides as an output an estimated pressure, Pm, in the patient interface 3000.

### 4.3.3.1.2 Vent Flow Rate Estimation

In one form of the present technology, a vent flow rate estimation algorithm 4314 receives as an input an estimated pressure, Pm, in the patient interface 3000 and estimates a vent flow rate of air, Qv, from a vent 3400 in a patient interface 3000.

### 4.3.3.1.3 Leak Flow Rate Estimation

In one form of the present technology, a leak flow rate estimation algorithm 4316 receives as an input a total flow rate Qt and a vent flow rate Qv, and estimates a leak flow rate Ql. In one form, the leak flow rate estimation algorithm 4316 estimates the leak flow rate Ql by calculating an average of the difference between the total flow rate and the vent flow rate Qv over a period sufficiently long to include several breathing cycles, e.g., about 10 seconds.

In one form, the leak flow estimation algorithm 4316 receives as an input a total flow rate Qt, a vent flow rate Qv, and an estimated pressure, Pm, in the patient interface 3000, and estimates a leak flow rate Ql by calculating a leak conductance, and determining a leak flow rate Ql to be a function of leak conductance and the pressure Pm. Leak conductance may be calculated as the quotient of low-pass filtered non-vent flow rate equal to the difference between total flow rate Qt and vent flow rate Qv, and low-pass filtered square root of pressure Pm, where the low-pass filter time constant has a value sufficiently long to include several breathing cycles, e.g., about 10 seconds. The leak flow rate Ql may be estimated as the product of leak conductance and a function of pressure, Pm.

### 4.3.3.1.4 Respiratory Flow Rate Estimation

In one form of the present technology, a respiratory flow rate estimation algorithm 4317 receives as an input a total flow rate, Qt, a vent flow rate, Qv, and a leak flow rate, Ql, and estimates a respiratory flow rate of air, Qr, to the patient, by subtracting the vent flow rate Qv and the leak flow rate Ql from the total flow rate Qt.

In other forms of the present technology, the respiratory flow estimation algorithm 4317 provides a value that acts as a proxy for the respiratory flow rate Qr. Possible proxies for respiratory flow rate include:
Respiratory movement of the chest of the patient 1000
Current drawn by the pressure generator 4140
Motor speed of the pressure generator 4140
Trans-thoracic impedance of the patient 1000

The respiratory flow rate proxy value may be provided by a transducer 4270 in the RPT device 4000, e.g., the motor speed sensor 4276, or a sensor external to the RPT device 4000, such a respiratory movement sensor or a trans-thoracic impedance sensor.

### 4.3.3.1.5 Ventilation Determination

In one form of the present technology, a ventilation determination algorithm 4311 receives an input a respiratory flow rate Qr, and determines a measure Vent indicative of current patient ventilation.

In some implementations, the ventilation determination algorithm 4311 determines a measure of ventilation Vent that is an estimate of actual patient ventilation.

In one such implementation, the measure of ventilation Vent is half the absolute value of respiratory flow, Qr, optionally filtered by low-pass filter such as a second order Bessel low-pass filter with a corner frequency of 0.11 Hz.

In one such implementation, the measure of ventilation Vent is an estimate of gross alveolar ventilation (i.e. non-anatomical-deadspace ventilation). This requires an estimate of anatomical deadspace. One can use the patient's height (or arm-span in cases of severe skeletal deformity) as a good predictor of anatomical deadspace. Gross alveolar ventilation is then equal to a measure of actual patient ventilation, e.g., determined as above, less the product of the estimated anatomical deadspace and the estimated spontaneous respiratory rate Rs.

In other implementations, the ventilation determination algorithm 4311 determines a measure of ventilation Vent that is broadly proportional to actual patient ventilation. One such implementation estimates peak respiratory flow rate Qpeak over the inspiratory portion of the cycle. This and many other procedures involving sampling the respiratory flow rate Qr produce measures which are broadly proportional to ventilation, provided the flow rate waveform shape does not vary very much (here, the shape of two breaths is taken to be similar when the flow rate waveforms of the breaths normalised in time and amplitude are similar). Some simple examples include the median positive respiratory flow rate, the median of the absolute value of respiratory flow rate, and the standard deviation of flow rate. Arbitrary linear combinations of arbitrary order statistics of the absolute value of respiratory flow rate using positive coefficients, and even some using both positive and negative coefficients, are approximately proportional to ventilation. Another example is the mean of the respiratory flow rate in the middle K proportion (by time) of the inspiratory portion, where 0<K<1. There is an arbitrarily large number of measures that are exactly proportional to ventilation if the flow rate waveform shape is constant.

In other forms, the ventilation determination algorithm 4311 determines a measure Vent of ventilation that is not based on respiratory flow rate Qr, but is a proxy for the current patient ventilation, such as oxygen saturation (SaO₂), or partial pressure of carbon dioxide (PCO₂), obtained from suitable sensors attached to the patient 1000.

### 4.3.3.1.6 Target Ventilation Determination

In one form of the present technology, a central controller 4230 takes as input the measure of current ventilation, Vent, and executes one or more target ventilation determination algorithms 4313 for the determination of a target value Vtgt for the measure of ventilation.

In some forms of the present technology, there is no target ventilation determination algorithm 4313, and the target ventilation Vtgt is predetermined, for example by hard-coding during configuration of the RPT device 4000 or by manual entry through the input device 4220.

In other forms of the present technology, such as adaptive servo-ventilation (ASV) therapy (described below), the target ventilation determination algorithm 4313 computes the target ventilation Vtgt from a value Vtyp indicative of the typical recent ventilation of the patient 1000.

In some forms of adaptive servo-ventilation therapy, the target ventilation Vtgt is computed as a high proportion of, but less than, the typical recent ventilation Vtyp. The high proportion in such forms may be in the range (80%, 100%), or (85%, 95%), or (87%, 92%).

In other forms of adaptive servo-ventilation therapy, the target ventilation Vtgt is computed as a slightly greater than unity multiple of the typical recent ventilation Vtyp.

The typical recent ventilation Vtyp is the value around which the distribution of the measure of current ventilation Vent over multiple time instants over some predetermined timescale tends to cluster, that is, a measure of the central tendency of the measure of current ventilation over recent history. In one implementation of the target ventilation determination algorithm 4313, the recent history is of the order of several minutes, but in any case should be longer than the timescale of Cheyne-Stokes waxing and waning cycles. The target ventilation determination algorithm 4313 may use any of the variety of well-known measures of central tendency to determine the typical recent ventilation Vtyp from the measure of current ventilation, Vent. One such measure is the output of a low-pass filter on the measure of current ventilation Vent, with time constant equal to one hundred seconds.

### 4.3.3.1.7 Respiratory Rate Estimation

In one form of the present technology, a respiratory rate estimation algorithm 4318 receives as an input a respiratory flow rate, Qr, to the patient 1000, and produces an estimate of the spontaneous respiratory rate Rs of the patient.

The respiratory rate estimation algorithm 4318 may estimate the spontaneous respiratory rate Rs over periods when the patient 1000 is breathing spontaneously, i.e., when the RPT device 4000 is not delivering “backup breaths” (described below). In some forms of the present technology, the respiratory rate estimation algorithm 4318 estimates the respiratory rate over periods when servo-assistance (defined as pressure support minus minimum pressure support) is low, in one implementation less than 4 cmH₂O, as such periods are more likely to reflect spontaneous respiratory effort.

In some forms of the present technology, the respiratory rate estimation algorithm 4318 estimates the respiratory rate over periods of asleep breathing, since the respiratory rate during these periods may be substantially different from the respiratory rate during wake. Anxiety typically results in a higher respiratory rate than that prevailing during sleep. When patients focus on their own breathing process, their respiratory rates are typically lower than those during normal wakefulness or during sleep. Techniques such as described in Patent Application no. PCT/AU2010/000894, published as WO 2011/006199, the entire disclosure of which is hereby incorporated herein by reference, may be used to identify periods of awake breathing from the respiratory flow rate, Qr.

In some forms of the present technology, the respiratory rate estimation algorithm 4318 estimates the spontaneous respiratory rate Rs as the reciprocal of one of a variety of well-known statistical measures of central tendency of breath duration Ttot during the period of interest. In such measures it is desirable to reject, or at least be robust to, outliers. One such measure, trimmed mean, in which the lower and upper K proportions of the sorted breath durations are discarded and the mean calculated on the remaining breath durations, is robust to outliers. For example, when K is 0.25, this amounts to discarding the upper and lower quartiles of breath duration Ttot. The median is another robust measure of central tendency, though this can occasionally give unsatisfactory results when the distribution is strongly bimodal. A simple mean may also be employed as a measure of central tendency, though it is sensitive to outliers. An initial interval filtering stage, in which contiguous time intervals corresponding to implausible respiratory rates (e.g., greater than 45 breaths/minute or less than 6 breaths/minute) are excluded as outliers from the mean calculation, may be employed. Other filtering mechanisms which may be used alone or in combination with interval filtering are to exclude any breaths that are not part of a sequence of N successive spontaneous breaths, where N is some small integer (e.g., 3), and to exclude the early and late breaths of a sequence of successive spontaneous breaths, e.g., to exclude the first and last breaths of a sequence of four breaths. The rationale for the latter mechanism is that the first and the last breaths in particular, and the early and late breaths in general, of a sequence of spontaneous breaths may be atypical; for example, the first spontaneous breath may occur as a result of an arousal, and the last spontaneous breath may be longer because of the decreasing respiratory drive which results in the backup breath which ends the sequence of spontaneous breaths.

In some forms of the present technology, the respiratory rate estimation algorithm 4318 makes an initial estimate of the spontaneous respiratory rate Rs using an initial period of estimation, to enable the subsequent processing in the therapy engine module 4320 to begin, and then continuously updates the estimate of the spontaneous respiratory rate Rs using a period of estimation that is longer than the initial period of estimation, to improve statistical robustness. For example, the initial period of estimation may be 20 minutes of suitable spontaneous breaths, but the period of estimation may then progressively increase up to some maximum duration, for example 8 hours. Rather than a rolling window of this duration being used for this estimation, low-pass filters on breath duration may be used, with progressively longer response times (more precisely, progressively lower corner frequencies) as the session proceeds.

In some forms, a suitably processed short-term (e.g., 10-minute) measure of central tendency, such as trimmed mean, may be input to a suitable low-pass filter to give an estimate Rs which changes on the time scale of hours or longer. This has the advantage that potentially large amounts of breath duration data do not need to be stored and processed, as might occur if a trimmed mean needs to be calculated on a moving window of breath duration data lasting hours or days.

In some forms of the present technology, respiratory rates measured over short periods of time, and in particular over one breath, may also be used instead of breath duration in the above-described measures of central tendency, giving generally similar but not identical results.

### 4.3.3.1.8 Backup Rate Determination

In one form of the present technology, a backup rate determination algorithm 4319 receives as input a spontaneous respiratory rate estimate Rs provided by the respiratory rate estimation algorithm 4318 and returns a “backup rate” Rb. The backup rate Rb is the rate at which the RPT device 4000 will deliver backup breaths, i.e., continue to provide ventilatory support, to a patient 1000 in the absence of significant spontaneous respiratory effort.

In one form of the pre-processing module 4310, there is no backup rate determination algorithm 4319, and the backup rate Rb is instead provided manually to the RPT device 4000, e.g., via the input device 4220, or hard-coded at the time of configuration of the RPT device 4000.

In one form, known as adaptive backup rate, the backup rate determination algorithm 4319 determines the backup rate Rb as a function of the spontaneous respiratory rate Rs. In one implementation, the function determines the backup rate Rb as the spontaneous respiratory rate Rs minus a constant such as 2 breaths per minute. In another implementation, the function determines the backup rate Rb as the spontaneous respiratory rate Rs multiplied by a constant that is slightly less than unity.

In one form, known as variable backup rate, the backup rate determination algorithm 4319 determines the backup rate Rb as a function of time. The backup rate Rb is initialised to a value known as the spontaneous backup rate (SBR) that is some fraction of a final target backup rate, known as the sustained timed backup rate (STBR). The fraction may be two thirds, or three quarters, or other positive values less than one. The SBR is the reciprocal of the timeout period to a backup breath when the most recent inspiration was a spontaneous (i.e., patent-triggered) breath. The STBR may be predetermined (e.g., by manual entry or hard-coding as described above) or set to some typical respiratory rate such as 15 bpm. Over time elapsed since the previous spontaneous breath, the backup rate Rb is increased from the SBR towards the STBR. The increase may be according to a predetermined profile, such as a series of steps, or a continuous linear profile. The profile is chosen such that the backup rate Rb reaches the STBR after a predetermined interval. The interval may be measured in units of time, such as 30 seconds, or relative to the patient's respiration, such as 5 breaths.

In some forms of variable backup rate, the predetermined interval over which the backup rate Rb increases from the SBR towards the STBR may be a function of the adequacy of current ventilation. In one implementation, suitable for servo-ventilation in which a target value Vtgt exists for the measure of ventilation, the backup rate approaches the STBR faster to the extent that current measure of ventilation Vent is less than the target ventilation Vtgt.

In one form of variable backup rate, known as adaptive variable backup rate, the backup rate determination algorithm 4319 determines the backup rate Rb as a function of the current estimated spontaneous respiratory rate Rs provided by the respiratory rate estimation algorithm 4318, as well as a function of time. As in variable backup rate determination, adaptive variable backup rate determination increases the backup rate Rb from the SBR towards the STBR over a predetermined interval that may be a function of the adequacy of current ventilation. The STBR may be initialised to a standard respiratory rate, such as 15 bpm. Once a reliable estimate of spontaneous respiratory rate Rs is available from the respiratory rate estimation algorithm 4318, the STBR may be set to the current estimated spontaneous respiratory rate Rs multiplied by some constant. The SBR may be set to some fraction of the STBR, as in variable backup rate. In one form, the fraction, for example two thirds, can be set to a lower value, such as 0.55, during the initial period of estimation of the spontaneous respiratory rate Rs, to accommodate occasional long breath durations in patients with relatively low respiratory rates, such as 12 breaths per minute.

In some forms, the constant by which the current estimated spontaneous respiratory rate Rs is multiplied to obtain the STBR may be slightly higher than 1, e.g., 1.1, to provide more aggressive ventilation during apneas, which may be desirable in short apneas. The constant may be somewhat lower than 1, e.g., 0.8, particularly if difficulty in resynchronisation with the patient on the return of patient effort turns out to be a problem in a particular patient. Lower backup rates make resynchronisation easier, by lengthening the expiratory pause, during which resynchronisation commonly occurs.

### 4.3.3.2 Therapy Engine Module

In one form of the present technology, a therapy engine module 4320 receives as inputs one or more of a pressure, Pm, in a patient interface 3000, a respiratory flow rate of air to a patient, Qr, and an estimate Rs of the spontaneous respiratory rate, and provides as an output one or more therapy parameters. In various forms, the therapy engine module 4320 comprises one or more of the following algorithms: phase determination 4321, waveform determination 4322, inspiratory flow limitation determination 4324, apnea/hypopnea determination 4325, snore detection 4326, airway patency determination 4327, and therapy parameter determination 4329.

### 4.3.3.2.1 Phase Determination

In one form of the present technology, a phase determination algorithm 4321 receives as an input a signal indicative of respiratory flow, Qr, and provides as an output a phase Φ of a current breathing cycle of a patient 1000.

In some forms, known as discrete phase determination, the phase output Φ is a discrete variable. One implementation of discrete phase determination provides a bi-valued phase output Φ with values of either inhalation or exhalation, for example represented as values of 0 and 0.5 revolutions respectively, upon detecting the start of spontaneous inhalation and exhalation respectively. RPT devices 4000 that “trigger” and “cycle” effectively perform discrete phase determination, since the trigger and cycle points are the instants at which the phase changes from exhalation to inhalation and from inhalation to exhalation, respectively. In one implementation of bi-valued phase determination, the phase output (D is determined to have a discrete value of 0 (thereby “triggering” the RPT device 4000) when the respiratory flow rate Qr has a value that exceeds a positive threshold, and a discrete value of 0.5 revolutions (thereby “cycling” the RPT device 4000) when a respiratory flow rate Qr has a value that is more negative than a negative threshold.

Another implementation of discrete phase determination provides a tri-valued phase output Φ with a value of one of inhalation, mid-inspiratory pause, and exhalation.

In other forms, known as continuous phase determination, the phase output Φ is a continuous value, for example varying from 0 to 1 revolutions, or 0 to 2π radians. RPT devices 4000 that perform continuous phase determination may trigger and cycle when the continuous phase reaches 0 and 0.5 revolutions, respectively. In one implementation of continuous phase determination, a continuous value of phase Φ is determined using a fuzzy logic analysis of the respiratory flow rate Qr. A continuous value of phase determined in this implementation is often referred to as “fuzzy phase”. In one implementation of a fuzzy phase determination algorithm 4321, the following rules are applied to the respiratory flow rate Qr:
1. If the respiratory flow rate is zero and increasing fast then the phase is 0 revolutions.
2. If the respiratory flow rate is large positive and steady then the phase is 0.25 revolutions.
3. If the respiratory flow rate is zero and falling fast, then the phase is 0.5 revolutions.
4. If the respiratory flow rate is large negative and steady then the phase is 0.75 revolutions.
5. If the respiratory flow rate is zero and steady and the 5-second low-pass filtered absolute value of the respiratory flow rate is large then the phase is 0.9 revolutions.
6. If the respiratory flow rate is positive and the phase is expiratory, then the phase is 0 revolutions.
7. If the respiratory flow rate is negative and the phase is inspiratory, then the phase is 0.5 revolutions.
8. If the 5-second low-pass filtered absolute value of the respiratory flow rate is large, the phase is increasing at a steady rate equal to the patient's respiratory rate, low-pass filtered with a time constant of 20 seconds.

The output of each rule may be represented as a vector whose phase is the result of the rule and whose magnitude is the fuzzy extent to which the rule is true. The fuzzy extent to which the respiratory flow rate is “large”, “steady”, etc. is determined with suitable membership functions. The results of the rules, represented as vectors, are then combined by some function such as taking the centroid. In such a combination, the rules may be equally weighted, or differently weighted.

In another implementation of continuous phase determination, the inhalation time Ti and the exhalation time Te are first estimated from the respiratory flow rate Qr. The phase (D is then determined as the half the proportion of the inhalation time Ti that has elapsed since the previous trigger instant, or 0.5 revolutions plus half the proportion of the exhalation time Te that has elapsed since the previous cycle instant (whichever was more recent).

In some forms of the present technology, suitable for pressure support ventilation therapy (described below), the phase determination algorithm 4321 is configured to trigger even when the respiratory flow rate Qr is insignificant, such as during an apnea. As a result, the RPT device 4000 delivers “backup breaths” in the absence of spontaneous respiratory effort from the patient 1000. For such forms, known as spontaneous/timed (S/T) modes, the phase determination algorithm 4321 may make use of the backup rate Rb provided by the backup rate determination algorithm 4319.

A phase determination algorithm 4321 that uses “fuzzy phase” may implement S/T mode using the backup rate Rb by including a “momentum” rule in the fuzzy phase rules. The effect of the momentum rule is to carry the continuous phase forward from exhalation to inhalation at the backup rate Rb if there are no features of respiratory flow rate Qr that would otherwise carry the continuous phase forward through the other rules. In one implementation, the more it is true that the measure of ventilation Vent (described below) is well below a target value Vtgt for ventilation (also described below), the more highly the momentum rule is weighted in the combination. However, as a result of the rapid increase in pressure support in response to mild to moderate hypoventilation (with respect to the target ventilation), the ventilation may be quite close to the target ventilation. It is desirable that the momentum rule is given a low weighting when the ventilation is close to target, to allow the patient to breathe at rates significantly lower than the respiratory rate at other times (when the patient is not in a central apnea) without being unnecessarily pushed to breathe at a higher rate by the ventilator. However, when the momentum rule is given a low weighting when ventilation is above a value which is below but close to the target ventilation, adequate ventilation may easily be achieved at a relatively high pressure support at a rate well below the backup rate. It would be desirable for the backup breaths to be delivered at a higher rate, because this would enable the target ventilation to be delivered at a lower pressure support. This is desirable for a number of reasons, a key one of which is to diminish mask leak.

To summarise, in a fuzzy phase determination algorithm 4321 that implements S/T mode, there is a dilemma in choosing the weighting for the momentum rule incorporating the backup rate Rb: if it is too high, the patient may feel “pushed along” by the backup rate. If it is too low, the pressure support may be excessive. Hence it is desirable to provide methods of implementing S/T mode which do not rely on the momentum rule described above.

A phase determination algorithm 4321 (either discrete, or continuous without a momentum rule) may implement S/T mode using the backup rate Rb in a manner known as timed backup. Timed backup may be implemented as follows: the phase determination algorithm 4321 attempts to detect the start of inhalation due to spontaneous respiratory effort, for example by monitoring the respiratory flow rate Qr as described above. If the start of inhalation due to spontaneous respiratory effort is not detected within a period of time after the last trigger instant whose duration is equal to the reciprocal of the backup rate Rb (an interval known as the backup timing threshold), the phase determination algorithm 4321 sets the phase output (D to a value of inhalation (thereby triggering the RPT device 4000). Once the RPT device 4000 is triggered, and a backup breath begins to be delivered, the phase determination algorithm 4321 attempts to detect the start of spontaneous exhalation, for example by monitoring the respiratory flow rate Qr, upon which the phase output Φ is set to a value of exhalation (thereby cycling the RPT device 4000).

If the backup rate Rb is increased over time from the SBR to the STBR, as in a variable backup rate system described above, the backup timing threshold starts out longer and gradually becomes shorter. That is, the RPT device 4000 starts out less vigilant and gradually becomes more vigilant to lack of spontaneous respiratory effort as more backup breaths are delivered. Such an RPT device 4000 is less likely to make a patient feel “pushed along” if they would prefer to breathe at a lower than standard rate, while still delivering backup breaths when they are needed.

If the STBR in a variable backup rate system adapts to the patient's estimated spontaneous respiratory rate Rs, as in an adaptive variable backup rate system described above, the backup breaths will be delivered at a rate that adapts to the patient's own recent spontaneous respiratory efforts.

### 4.3.3.2.2 Waveform Determination

In one form of the present technology, the therapy control module 4330 controls a pressure generator 4140 to provide a treatment pressure Pt that varies as a function of phase (D of a breathing cycle of a patient according to a waveform template Π(Φ).

In one form of the present technology, a waveform determination algorithm 4322 provides a waveform template Π(Φ) with values in the range [0, 1] on the domain of phase values Φ provided by the phase determination algorithm 4321 to be used by the therapy parameter determination algorithm 4329.

In one form, suitable for either discrete or continuously-valued phase, the waveform template Π(Φ) is a square-wave template, having a value of 1 for values of phase up to and including 0.5 revolutions, and a value of 0 for values of phase above 0.5 revolutions. In one form, suitable for continuously-valued phase, the waveform template Π(Φ) comprises two smoothly curved portions, namely a smoothly curved (e.g., raised cosine) rise from 0 to 1 for values of phase up to 0.5 revolutions, and a smoothly curved (e.g., exponential) decay from 1 to 0 for values of phase above 0.5 revolutions. One example of such a “smooth and comfortable” waveform template is the “shark fin” waveform template, in which the rise is a raised cosine, and the smooth decay is quasi-exponential (so that the limit of Π as Φ approaches one revolution is precisely zero).

In some forms of the present technology, the waveform determination algorithm 4322 selects a waveform template Π(Φ) from a library of waveform templates, dependent on a setting of the RPT device 4000. Each waveform template Π(Φ) in the library may be provided as a lookup table of values Π against phase values Φ. In other forms, the waveform determination algorithm 4322 computes a waveform template Π(Φ) “on the fly” using a predetermined functional form, possibly parametrised by one or more parameters (e.g., time constant of an exponentially curved portion). The parameters of the functional form may be predetermined or dependent on a current state of the patient 1000.

In some forms of the present technology, suitable for discrete bi-valued phase of either inhalation (Φ=0 revolutions) or exhalation (Φ=0.5 revolutions), the waveform determination algorithm 4322 computes a waveform template Π “on the fly” as a function of both discrete phase Φ and time t measured since the most recent trigger instant (transition from exhalation to inhalation). In one such form, the waveform determination algorithm 4322 computes the waveform template Π(Φ, t) in two portions (inspiratory and expiratory) as follows:

$$Π ( Φ , t ) = \{ \begin{matrix}{Π}_{i} ( t ) , & Φ = 0 \\ {Π}_{e} ( t - {T}_{i} ) , & Φ = 0.5\end{matrix}$$

where Φᵢ(t) and Πₑ(t) are inspiratory and expiratory portions of the waveform template Π(Φ, t), and Ti is the inhalation time. In one such form, the inspiratory portion Πᵢ(t) of the waveform template is a smooth rise from 0 to 1 parametrised by a rise time, and the expiratory portion Πₑ(t) of the waveform template is a smooth fall from 1 to 0 parametrised by a fall time.

### 4.3.3.2.3 Determination of Inspiratory Flow Limitation

In one form of the present technology, a processor executes one or more algorithms 4324 for the detection of inspiratory flow limitation (partial obstruction).

In one form the algorithm 4324 receives as an input a respiratory flow rate signal Qr and provides as an output a metric of the extent to which the inspiratory portion of the breath exhibits inspiratory flow limitation.

In one form of the present technology, the inspiratory portion of each breath is identified based on the phase Φ estimated at each instant. For example, the inspiratory portion of the breath is the values of respiratory flow for which the phase Φ is less than or equal to 0.5. A number of evenly spaced points (for example, sixty-five), representing points in time, are interpolated by an interpolator along the inspiratory flow-time curve for each breath. The curve described by the points is then scaled by a scaler to have unity length (duration/period) and unity area to remove the effects of changing respiratory rate and depth. The scaled breaths are then compared in a comparator with a pre-stored template representing a normal unobstructed breath. Breaths deviating by more than a specified threshold (typically 1 scaled unit) at any time during the inspiration from this template, such as those due to coughs, sighs, swallows and hiccups, as determined by a test element, are rejected. For non-rejected data, a moving average of the first such scaled point is calculated by central controller 4230 for the preceding several inspiratory events. This is repeated over the same inspiratory events for the second such point, and so on. Thus, for example, sixty five scaled data points are generated by central controller 4230, and represent a moving average of the preceding several inspiratory events, e.g., three events. The moving average of continuously updated values of the (e.g., sixty five) points are hereinafter called the “scaled flow”, designated as Qs(t). Alternatively, a single inspiratory event can be utilised rather than a moving average.

From the scaled flow, two shape factors relating to the determination of partial obstruction may be calculated.

Shape factor 1 is the ratio of the mean of the middle (e.g., thirty-two) scaled flow points to the mean overall (e.g., sixty-five) scaled flow points. Where this ratio is in excess of unity, the breath will be taken to be normal. Where the ratio is unity or less, the breath will be taken to be obstructed. A ratio of about 1.17 is taken as a threshold between partially obstructed and unobstructed breathing, and equates to a degree of obstruction that would permit maintenance of adequate oxygenation in a typical user.

Shape factor 2 is calculated as the RMS deviation from unit scaled flow, taken over the middle (e.g., thirty two) points. An RMS deviation of about 0.2 units is taken to be normal. An RMS deviation of zero is taken to be a totally flow-limited breath. The closer the RMS deviation to zero, the breath will be taken to be more flow limited.

Shape factors 1 and 2 may be used as alternatives, or in combination. In other forms of the present technology, the number of sampled points, breaths and middle points may differ from those described above. Furthermore, the threshold values can other than those described.

### 4.3.3.2.4 Determination of Apneas and Hypopneas

In one form of the present technology, a central controller 4230 executes one or more algorithms 4325 for the detection of apneas and/or hypopneas.

In one form, the one or more apnea/hypopnea detection algorithms 4325 receive as an input a respiratory flow rate Qr and provide as an output a flag that indicates that an apnea or a hypopnea has been detected.

In one form, an apnea will be said to have been detected when a function of respiratory flow rate Qr falls below a flow threshold for a predetermined period of time. The function may determine a peak flow, a relatively short-term mean flow, or a flow intermediate of relatively short-term mean and peak flow, for example an RMS flow. The flow threshold may be a relatively long-term measure of flow.

In one form, a hypopnea will be said to have been detected when a function of respiratory flow rate Qr falls below a second flow threshold for a predetermined period of time. The function may determine a peak flow, a relatively short-term mean flow, or a flow intermediate of relatively short-term mean and peak flow, for example an RMS flow. The second flow threshold may be a relatively long-term measure of flow. The second flow threshold is greater than the flow threshold used to detect apneas.

In one form, such respiratory events may be characterized as central or obstructive based at least in part on the aforementioned finger sensor PPG based type detection.

### 4.3.3.2.5 Detection of Snore

In one form of the present technology, a central controller 4230 executes one or more snore detection algorithms 4326 for the detection of snore.

In one form, the snore detection algorithm 4326 receives as an input a respiratory flow rate signal Qr and provides as an output a metric of the extent to which snoring is present.

The snore detection algorithm 4326 may comprise a step of determining the intensity of the flow rate signal in the range of 30-300 Hz. The snore detection algorithm 4326 may further comprises a step of filtering the respiratory flow rate signal Qr to reduce background noise, e.g., the sound of airflow in the system from the blower 4142.

### 4.3.3.2.6 Determination of Airway Patency

In one form of the present technology, a central controller 4230 executes one or more algorithms 4327 for the determination of airway patency.

In one form, airway patency algorithm 4327 receives as an input a respiratory flow rate signal Qr, and determines the power of the signal in the frequency range of about 0.75 Hz and about 3 Hz. The presence of a peak in this frequency range is taken to indicate an open airway. The absence of a peak is taken to be an indication of a closed airway.

In one form, the frequency range within which the peak is sought is the frequency of a small forced oscillation in the treatment pressure Pt. In one implementation, the forced oscillation is of frequency 2 Hz with amplitude about 1 cmH₂O.

In one form, airway patency algorithm 4327 receives as an input a respiratory flow rate signal Qr, and determines the presence or absence of a cardiogenic signal. The absence of a cardiogenic signal is taken to be an indication of a closed airway.

### 4.3.3.2.7 Determination of Therapy Parameters

In some forms of the present technology, the central controller 4230 executes one or more therapy parameter determination algorithms 4329 for the determination of one or more therapy parameters using the values returned by one or more of the other algorithms in the therapy engine module 4320.

In one form of the present technology, the therapy parameter is an instantaneous treatment pressure Pt. In one implementation of this form, the therapy parameter determination algorithm 4329 determines the treatment pressure Pt using the equation

$$P t = A Π ( Φ ) + {P}_{0} \qquad ( 1 )$$

where:
   A is an amplitude,
   Φ is the current value of phase;
   Π(Φ) is the waveform template value (in the range 0 to 1) at the current value of phase, and
   P₀ is a base pressure.

If the waveform determination algorithm 4322 provides the waveform template Π(Φ) as a lookup table of values indexed by phase Φ, the therapy parameter determination algorithm 4329 applies equation (1) by locating the nearest lookup table entry to the current value Φ of phase returned by the phase determination algorithm 4321, or by interpolation between the two entries straddling the current value Φ of phase.

The values of the amplitude A and the base pressure P₀ may be set by the therapy parameter determination algorithm 4329 depending on the chosen pressure therapy mode in the manner described below.

### 4.3.3.3 Therapy Control Module

The therapy control module 4330 in accordance with one aspect of the present technology receives as inputs the therapy parameters from the therapy parameter determination algorithm 4329 of the therapy engine module 4320, and controls the pressure generator 4140 to deliver a flow of air in accordance with the therapy parameters.

In one form of the present technology, the therapy parameter is a treatment pressure Pt, and the therapy control module 4330 controls the pressure generator 4140 to deliver a flow of gas whose mask pressure Pm at the patient interface 3000 is equal to the treatment pressure Pt.

### 4.3.3.4 Detection of Fault Conditions

In one form of the present technology, a processor executes one or more methods 4340 for the detection of fault conditions. The fault conditions detected by the one or more methods may include at least one of the following:
Power failure (no power, or insufficient power)
Transducer fault detection
Failure to detect the presence of a component
Operating parameters outside recommended ranges (e.g., pressure, flow, temperature, PaO₂)
Failure of a test alarm to generate a detectable alarm signal.

Upon detection of the fault condition, the corresponding algorithm signals the presence of the fault by one or more of the following:
Initiation of an audible, visual &/or kinetic (e.g., vibrating) alarm
Sending a message to an external device
Logging of the incident

### 4.4 Humidifier

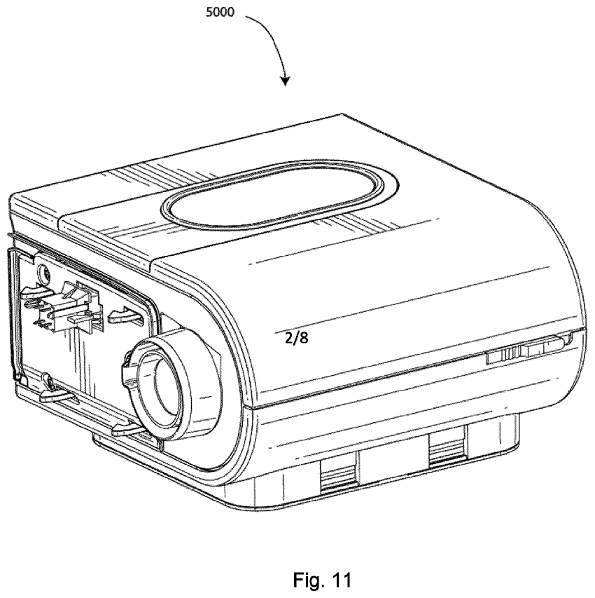

In one form of the present technology there is provided a humidifier 5000 (e.g., as shown in FIG. 11) to change the absolute humidity of air or gas for delivery to a patient relative to ambient air. Typically, the humidifier 5000 is used to increase the absolute humidity and increase the temperature of the flow of air (relative to ambient air) before delivery to the patient's airways.

### 4.5 Glossary

For the purposes of the present disclosure, in certain forms of the present technology, one or more of the following definitions may apply. In other forms of the present technology, alternative definitions may apply.

### 4.5.1 General

Air: In certain forms of the present technology, air may be taken to mean atmospheric air, and in other forms of the present technology air may be taken to mean some other combination of breathable gases, e.g., atmospheric air enriched with oxygen.

Respiratory Pressure Therapy (RPT): The delivery of a supply of air to the airways at a treatment pressure that is typically positive with respect to atmosphere.

Continuous Positive Airway Pressure (CPAP) therapy: Respiratory pressure therapy in which the treatment pressure is approximately constant through a breathing cycle of a patient. In some forms, the pressure at the entrance to the airways will be slightly higher during exhalation, and slightly lower during inhalation. In some forms, the pressure will vary between different breathing cycles of the patient, for example, being increased in response to detection of indications of partial upper airway obstruction, and decreased in the absence of indications of partial upper airway obstruction.

Patient: A person, whether or not they are suffering from a respiratory disease.

Automatic Positive Airway Pressure (APAP) therapy: CPAP therapy in which the treatment pressure is automatically adjustable, e.g., from breath to breath, between minimum and maximum limits, depending on the presence or absence of indications of SDB events.

### 4.5.2 Aspects of the Breathing Cycle

Apnea: According to some definitions, an apnea is said to have occurred when respiratory flow rate falls below a predetermined threshold for a duration, e.g., 10 seconds. An obstructive apnea will be said to have occurred when, despite patient effort, some obstruction of the airway does not allow air to flow. A central apnea will be said to have occurred when an apnea is detected that is due to a reduction in breathing effort, or the absence of breathing effort.

Breathing rate, or respiratory rate (Rs): The rate of spontaneous respiration of a patient, usually measured in breaths per minute.

Duty cycle: The ratio of inhalation time, Ti to total breath duration, Ttot.

Effort (breathing): The work done by a spontaneously breathing person attempting to breathe.

Expiratory portion of a breathing cycle: The period from the start of expiratory flow to the start of inspiratory flow.

Flow limitation: The state of affairs in a patient's respiration where an increase in effort by the patient does not give rise to a corresponding increase in flow. Where flow limitation occurs during an inspiratory portion of the breathing cycle it may be described as inspiratory flow limitation. Where flow limitation occurs during an expiratory portion of the breathing cycle it may be described as expiratory flow limitation.

Hypopnea: A reduction in flow, but not a cessation of flow. In one form, a hypopnea may be said to have occurred when there is a reduction in flow below a threshold for a duration. In one form in adults, the following either of the following may be regarded as being hypopneas:
(i) a 30% reduction in patient breathing for at least 10 seconds plus an associated 4% desaturation; or
(ii) a reduction in patient breathing (but less than 50%) for at least 10 seconds, with an associated desaturation of at least 3% or an arousal.

Inspiratory portion of a breathing cycle: The period from the start of inspiratory flow to the start of expiratory flow will be taken to be the inspiratory portion of a breathing cycle.

Patency (airway): The degree of the airway being open, or the extent to which the airway is open. A patent airway is open. Airway patency may be quantified, for example with a value of one (1) being patent, and a value of zero (0), being closed.

Positive End-Expiratory Pressure (PEEP): The pressure above atmosphere in the lungs that exists at the end of expiration.

Peak flow rate (Qpeak): The maximum value of flow during the inspiratory portion of the respiratory flow rate waveform.

Respiratory flow/airflow rate, patient flow/airflow rate (Qr): These synonymous terms may be understood to refer to the RPT device's estimate of respiratory airflow rate, as opposed to “true respiratory flow rate” or “true respiratory airflow rate”, which is the actual respiratory flow rate experienced by the patient, usually expressed in litres per minute.

Tidal volume (Vt): The volume of air inhaled or exhaled during normal breathing, when extra effort is not applied.

Inhalation Time (Ti): The duration of the inspiratory portion of the respiratory flow rate waveform.

Exhalation Time (Te): The duration of the expiratory portion of the respiratory flow rate waveform.

(total) Time, or breath duration (Ttot): The total duration between the start of the inspiratory portion of one respiratory flow rate waveform and the start of the inspiratory portion of the following respiratory flow rate waveform.

Upper airway obstruction (UAO): includes both partial and total upper airway obstruction. This may be associated with a state of flow limitation, in which the flow rate increases only slightly or may even decrease as the pressure difference across the upper airway increases (Starling resistor behaviour).

Ventilation (Vent): A measure of the total amount of gas being exchanged by the patient's respiratory system. Measures of ventilation may include one or both of inspiratory and expiratory flow, per unit time. When expressed as a volume per minute, this quantity is often referred to as “minute ventilation”. Minute ventilation is sometimes given simply as a volume, understood to be the volume per minute.

### 4.5.3 RPT Device Parameters

Flow rate: The instantaneous volume (or mass) of air delivered per unit time. While flow rate and ventilation have the same dimensions of volume or mass per unit time, flow rate is measured over a much shorter period of time. Flow may be nominally positive for the inspiratory portion of a breathing cycle of a patient, and hence negative for the expiratory portion of the breathing cycle of a patient. In some cases, a reference to flow rate will be a reference to a scalar quantity, namely a quantity having magnitude only. In other cases, a reference to flow rate will be a reference to a vector quantity, namely a quantity having both magnitude and direction. Flow rate will be given the symbol Q. ‘Flow rate’ is sometimes shortened to simply ‘flow’. Total flow rate, Qt, is the flow of air leaving the RPT device. Vent flow rate, Qv, is the flow of air leaving a vent to allow washout of exhaled gases. Leak flow rate, Ql, is the flow rate of unintentional leak from a patient interface system. Respiratory flow rate, Qr, is the flow of air that is received into the patient's respiratory system.

Leak: The word leak will be taken to be an unintended flow of air. In one example, leak may occur as the result of an incomplete seal between a mask and a patient's face. In another example leak may occur in a swivel elbow to the ambient.

Pressure: Force per unit area. Pressure may be measured in a range of units, including cmH₂O, g-f/cm², hectopascal. 1 cmH₂O is equal to 1 g-f/cm² and is approximately 0.98 hectopascal. In this specification, unless otherwise stated, pressure is given in units of cmH₂O. The pressure in the patient interface (mask pressure) is given the symbol Pm, while the treatment pressure, which represents a target value to be achieved by the mask pressure Pm at the current instant of time, is given the symbol Pt.

### 4.5.4 Terms for Ventilators

Adaptive Servo-Ventilator (ASV): A servo-ventilator that has a changeable rather than a fixed target ventilation. The changeable target ventilation may be learned from some characteristic of the patient, for example, a respiratory characteristic of the patient.

Backup rate: A parameter of a ventilator that establishes the respiratory rate (typically in number of breaths per minute) that the ventilator will deliver to the patient, if not triggered by spontaneous respiratory effort.

Cycled: The termination of a ventilator's inspiratory phase. When a ventilator delivers a breath to a spontaneously breathing patient, at the end of the inspiratory portion of the breathing cycle, the ventilator is said to be cycled to stop delivering the breath.

Expiratory positive airway pressure (EPAP): a base pressure, to which a pressure varying within the breath is added to produce the desired mask pressure which the ventilator will attempt to achieve at a given time.

End expiratory pressure (EEP): Desired mask pressure which the ventilator will attempt to achieve at the end of the expiratory portion of the breath. If the pressure waveform template Π(Φ) is zero-valued at the end of expiration, i.e., Π(Φ)=0 when Φ=1, the EEP is equal to the EPAP.

IPAP: desired mask pressure which the ventilator will attempt to achieve during the inspiratory portion of the breath.

Pressure support: A number that is indicative of the increase in pressure during ventilator inspiration over that during ventilator expiration, and generally means the difference in pressure between the maximum value during inspiration and the base pressure (e.g., PS=IPAP−EPAP). In some contexts pressure support means the difference which the ventilator aims to achieve, rather than what it actually achieves.

Servo-ventilator: A ventilator that measures patient ventilation, has a target ventilation, and which adjusts the level of pressure support to bring the patient ventilation towards the target ventilation.

Servo-assistance: Pressure support minus minimum pressure support.

Spontaneous/Timed (S/T): A mode of a ventilator or other device that attempts to detect the initiation of a breath of a spontaneously breathing patient. If however, the device is unable to detect a breath within a predetermined period of time, the device will automatically initiate delivery of the breath.

Swing: Equivalent term to pressure support.

Triggered: When a ventilator delivers a breath of air to a spontaneously breathing patient, it is said to be triggered to do so at the initiation of the inspiratory portion of the breathing cycle by the patient's efforts.

Typical recent ventilation: The typical recent ventilation Vtyp is the value around which recent measures of ventilation over some predetermined timescale tend to cluster, that is, a measure of the central tendency of the measures of ventilation over recent history.

Ventilator: A mechanical device that provides pressure support to a patient to perform some or all of the work of breathing.

### 4.5.5 Anatomy of the Respiratory System

Diaphragm: A sheet of muscle that extends across the bottom of the rib cage. The diaphragm separates the thoracic cavity, containing the heart, lungs and ribs, from the abdominal cavity. As the diaphragm contracts the volume of the thoracic cavity increases and air is drawn into the lungs.

Larynx: The larynx, or voice box houses the vocal folds and connects the inferior part of the pharynx (hypopharynx) with the trachea.

Lungs: The organs of respiration in humans. The conducting zone of the lungs contains the trachea, the bronchi, the bronchioles, and the terminal bronchioles. The respiratory zone contains the respiratory bronchioles, the alveolar ducts, and the alveoli.

Nasal cavity: The nasal cavity (or nasal fossa) is a large air filled space above and behind the nose in the middle of the face. The nasal cavity is divided in two by a vertical fin called the nasal septum. On the sides of the nasal cavity are three horizontal outgrowths called nasal conchae (singular “concha”) or turbinates. To the front of the nasal cavity is the nose, while the back blends, via the choanae, into the nasopharynx.

Pharynx: The part of the throat situated immediately inferior to (below) the nasal cavity, and superior to the oesophagus and larynx. The pharynx is conventionally divided into three sections: the nasopharynx (epipharynx) (the nasal part of the pharynx), the oropharynx (mesopharynx) (the oral part of the pharynx), and the laryngopharynx (hypopharynx).

### 4.6 Other Remarks

A portion of the disclosure of this patent document contains material which is subject to copyright protection. The copyright owner has no objection to the facsimile reproduction by anyone of the patent document or the patent disclosure, as it appears in the Patent and Trademark Office patent file or records, but otherwise reserves all copyright rights whatsoever.

Unless the context clearly dictates otherwise and where a range of values is provided, it is understood that each intervening value, to the tenth of the unit of the lower limit, between the upper and lower limit of that range, and any other stated or intervening value in that stated range is encompassed within the technology. The upper and lower limits of these intervening ranges, which may be independently included in the intervening ranges, are also encompassed within the technology, subject to any specifically excluded limit in the stated range. Where the stated range includes one or both of the limits, ranges excluding either or both of those included limits are also included in the technology.

Furthermore, where a value or values are stated herein as being implemented as part of the technology, it is understood that such values may be approximated, unless otherwise stated, and such values may be utilized to any suitable significant digit to the extent that a practical technical implementation may permit or require it.

Unless defined otherwise, all technical and scientific terms used herein have the same meaning as commonly understood by one of ordinary skill in the art to which this technology belongs. Although any methods and materials similar or equivalent to those described herein can also be used in the practice or testing of the present technology, a limited number of the exemplary methods and materials are described herein.

When a particular material is identified as being preferably used to construct a component, obvious alternative materials with similar properties may be used as a substitute. Furthermore, unless specified to the contrary, any and all components herein described are understood to be capable of being manufactured and, as such, may be manufactured together or separately.

It must be noted that as used herein and in the appended claims, the singular forms “a”, “an”, and “the” include their plural equivalents, unless the context clearly dictates otherwise.

All publications mentioned herein are incorporated by reference to disclose and describe the methods and/or materials which are the subject of those publications. The publications discussed herein are provided solely for their disclosure prior to the filing date of the present application. Nothing herein is to be construed as an admission that the present technology is not entitled to antedate such publication by virtue of prior invention. Further, the dates of publication provided may be different from the actual publication dates, which may need to be independently confirmed.

Moreover, in interpreting the disclosure, all terms should be interpreted in the broadest reasonable manner consistent with the context. In particular, the terms “comprises” and “comprising” should be interpreted as referring to elements, components, or steps in a non-exclusive manner, indicating that the referenced elements, components, or steps may be present, or utilized, or combined with other elements, components, or steps that are not expressly referenced.

The subject headings used in the detailed description are included only for the ease of reference of the reader and should not be used to limit the subject matter found throughout the disclosure or the claims. The subject headings should not be used in construing the scope of the claims or the claim limitations.

Although the technology herein has been described with reference to particular embodiments, it is to be understood that these embodiments are merely illustrative of the principles and applications of the technology. In some instances, the terminology and symbols may imply specific details that are not required to practice the technology. For example, although the terms “first” and “second” may be used, unless otherwise specified, they are not intended to indicate any order but may be utilised to distinguish between distinct elements. Furthermore, although process steps in the methodologies may be described or illustrated in an order, such an ordering is not required. Those skilled in the art will recognize that such ordering may be modified and/or aspects thereof may be conducted concurrently or even synchronously.

It is therefore to be understood that numerous modifications may be made to the illustrative embodiments and that other arrangements may be devised without departing from the spirit and scope of the technology.

Although the present invention has been illustrated by reference to specific embodiments, it will be apparent to those skilled in the art that the invention is not limited to the details of the foregoing illustrative embodiments, and that the present technology may be embodied with various changes and modifications without departing from the scope thereof. The present examples are therefore to be considered in all respects as illustrative and not restrictive, the scope of the technology being indicated by the appended claims rather than by the foregoing description, and all changes which come within the meaning and range of equivalency of the claims are therefore intended to be embraced therein. In other words, it is contemplated to cover any and all modifications, variations or equivalents that fall within the scope of the basic underlying principles and whose essential attributes are claimed in this patent application. It will furthermore be understood by the reader of this patent application that the words “comprising” or “comprise” do not exclude other elements or steps, that the words “a” or “an” do not exclude a plurality, and that a single element, such as a computer system, a processor, or another integrated unit may fulfil the functions of several means recited in the claims. Any reference signs in the claims shall not be construed as limiting the respective claims concerned. The terms “first”, “second”, third”, “a”, “b”, “c”, and the like, when used in the description or in the claims are introduced to distinguish between similar elements or steps and are not necessarily describing a sequential or chronological order. Similarly, the terms “top”, “bottom”, “over”, “under”, and the like are introduced for descriptive purposes and not necessarily to denote relative positions. It is to be understood that the terms so used are interchangeable under appropriate circumstances and embodiments of the technology are capable of operating according to the present technology in other sequences, or in orientations different from the one(s) described or illustrated above.

1. A memory card comprising:
a card interface configured to communicate with a medical device;
a non-volatile memory configured to store authentication information to join a wireless network, the non-volatile memory being configured to store therapy data provided by the medical device;
a network interface including at least one wireless communication transceiver; and
one or more processors configured to:
instruct the at least one wireless communication transceiver to join the wireless network using the authentication information; and
instruct the at least one wireless communication transceiver to access and wirelessly transmit the therapy data stored in the memory to a remote server via the wireless network.

2. The memory card of claim 1, wherein the authentication information is received from the medical device via the card interface, and stored in the non-volatile memory after being received from the medical device.

3. The memory card of claim 1, wherein the at least one wireless communication transceiver is configured to wirelessly receive the authentication information from a wireless device.

4. The memory card of claim 1, wherein the at least one wireless communication transceiver includes a wireless fidelity (Wi-Fi) communication transceiver.

5. The memory card of claim 1, wherein the one or more processors are configured to encrypt the data provided by the medical device, and instruct the at least one wireless communication transceiver to wirelessly transmit the encrypted data to the remote server via the wireless network.

6. The memory card of claim 1, wherein the card interface is configured to be physically and operably engaged with the medical device.

7. The memory card of claim 1, wherein the authentication information to join the wireless network includes a wireless network name and a password to access the wireless network.

8. The memory card of claim 1, wherein the one or more processors are configured to instruct the at least one wireless communication transceiver to access the remote server using credential information.

9. The memory card of claim 8, wherein the credential information includes a device identifier of the medical device.

10. The memory card of claim 8, wherein the credential information is obtained in any of the following manners:
receiving the credential information from the medical device via the card interface;
wirelessly receiving the credential information from a wireless device; and
retrieving the credential information from a firmware of the memory card.

11. The memory card of claim 1, wherein the one or more processors are configured to:
detect new data being written into the non-volatile memory; and
instruct the at least one wireless communication transceiver to wirelessly transmit the new data to the remote server via the wireless network.

12. A method comprising:
receiving, via a card interface of a memory card, therapy data provided by a medical device;
storing, in a non-volatile memory of the memory card, the received therapy data;
instructing, by one or more processors of the memory card, at least one wireless communication transceiver to join a wireless network using authentication information; and
instructing, by the one or more processors of the memory card, the at least one wireless communication transceiver to wirelessly transmit the therapy data stored in the non-volatile memory to a remote server via the wireless network.

13. The method of claim 12, further comprising receiving the authentication information from the medical device via the card interface.

14. The method of claim 12, further comprising wirelessly receiving, by the at least one wireless communication transceiver, the authentication information from a wireless device.

15. The method of claim 12, wherein the at least one wireless communication transceiver includes a wireless fidelity (Wi-Fi) communication transceiver.

16. The method of claim 12, further comprising:
instructing, by the one or more processors of the memory card, the at least one wireless communication transceiver to access the remote server using credential information.

17. The method of claim 16, further comprising:
receiving the credential information from the medical device via the card interface;
wirelessly receiving the credential information from a wireless device; or
retrieving the credential information from a firmware of the memory card.

18. A method of data reading, comprising:
reading a first barcode output by a medical device, the first barcode encoding information of the medical device;
registering, by one or more processors, the medical device with a remote server based on the first barcode;
reading a second barcode output by the medical device, the second barcode encoding therapy data recorded by the medical device; and
wirelessly transmitting the second barcode or the therapy data to the remote server.

19. The method of claim 18, further comprising:
decoding the first barcode to obtain the information of the medical device; and
transmitting, by a wireless transceiver, the decoded information of the medical device to the remote server.

20. The method of claim 18, wherein the first barcode is further configured to encode an encryption key.

21. The method of claim 18, wherein the information of the medical device includes at least one of the following: a device identifier and/or one or more device settings.

22. The method of claim 21, wherein the device settings include one or more of the following: therapy mode, maximum pressure, minimum pressure, and expiratory pressure relief (EPR) pressure.

23. The method of claim 20, further comprising:
decoding the first barcode to obtain the information of the medical device and the encryption key;
encrypting the information of the medical device by using the encryption key; and
transmitting, by a wireless transceiver, the encrypted information of the medical device to the remote server.

24. The method of claim 18, wherein each of the first barcode and the second barcode is a two-dimensional code.

25. The method of claim 18, further comprising:
decoding the second barcode to obtain the therapy data recorded by the medical device; and
wirelessly transmitting the therapy data to the remote server after decoding the second barcode.

26. The method of claim 18, further comprising:
displaying a prompt to a user to scan the second barcode according to a predetermined schedule.

27. The method of claim 18, further comprising:
determining, by the one or more processors, whether the second barcode has been scanned according to a predetermined schedule; and
displaying a prompt to a user to scan the second barcode when the second barcode has not been scanned according to the predetermined schedule.

28. The method of claim 18, wherein the medical device is a respiratory pressure medical device.

29. The method of claim 18, wherein the therapy data includes one or more of the following: one or more respiratory parameters of a user as collected by the medical device, usage data of the medical device, and one or more device settings of the medical device.

30. The method of claim 20, further comprising:
decoding the second barcode to obtain the therapy data recorded by the medical device;
encrypting the therapy data by using the encryption key obtained from the first barcode; and
wirelessly transmitting the encrypted therapy data to the remote server.

31. A method for reporting therapy data of a medical device to a remote server, comprising:
transforming information of the medical device to a first barcode;
outputting, for display, the first barcode for registering the medical device with a remote server;
transforming therapy data recorded by the medical device to a second barcode; and
outputting, for display, the second barcode for transmitting the therapy data to the remote server.

32. The method of claim 31, wherein the information of the medical device transformed to the first barcode includes one or more of the following: a serial number, one or more device settings, and an encryption key of the medical device.

33. The method of claim 32, wherein the device settings include one or more of the following: therapy mode, maximum pressure, minimum pressure, and expiratory pressure relief (EPR) pressure.

34. The method of claim 31, wherein each of the first barcode and the second barcode is a two-dimensional code.
